(19) United States
(12) Patent Application Publication
(10) Pub. No.: US 2022/0290140 A1
(43) Pub. Date: Sep. 15, 2022

(54) TRANS-CYCLOOCTENE LABELED ANTISENSE OLIGONUCLEOTIDES, RADIO LABELED TETRAZINE AND METHODS

(71) Applicants: Biogen MA Inc., Cambridge, MA (US); Ionis Pharmaceuticals, Inc., Carlsbad, CA (US)

(72) Inventors: Brendon Ellery COOK, Medford, MA (US); Maciej Adam KALISZCZAK, Belmont, MA (US); Laurent MARTARELLO, Lexington, MA (US); Nathan Edward GENUNG, Charlestown, MA (US); Sivaraman DANDAPANI, Wakefield, MA (US); Eric Edward SWAYZE, Encinitas, CA (US); William John DRURY, III, Oceanside, CA (US); Chrissa Ann DWYER, San Diego, CA (US)

(21) Appl. No.: 17/636,600

(22) PCT Filed: Aug. 19, 2020
(86) PCT No.: PCT/US2020/046984

§ 371 Date: Feb. 18, 2022

Related U.S. Application Data
(60) Provisional application No. 62/889,395, filed on Aug. 20, 2019.

(51) Int. Cl. C12N 15/113; A61K 47/54; A61K 51/04; C07F 9/165

(52) U.S. Cl. CPC C12N 15/113; A61K 47/549; A61K 47/555; A61K 51/0491; C07F 9/165; C12N 2310/113

(57) ABSTRACT

Featured are trans-cyclooctene and tetrazine compounds. Also provided are methods for evaluating the biodistribution and/or concentration of a biomolecule in a subject.

### CROSS-REFERENCE TO RELATED APPLICATIONS

This application claims the benefit of priority of U.S. Provisional Appl. No. 62/889,395, filed Aug. 20, 2019, the contents of which are incorporated by reference herein in their entirety.

### TECHNICAL FIELD

This disclosure relates generally to compositions and methods for assessing the distribution and/or concentrations of antisense oligonucleotides in a subject.

### BACKGROUND

Biomolecules such as antisense oligonucleotides (ASOs) have proven extremely efficacious in treating certain genetic diseases by binding their complementary mRNA sequence, and thereby reducing or preventing translation of aberrant genes. To ensure their proper distribution and kinetics, it is necessary to develop imaging modalities that are compatible with ASOs. While directly radiolabeled ASOs have proven effective, their application in human subjects has been limited by challenges relating to intrathecal administration of radiotracers and constraints on longer imaging time-points imposed by radioisotope half-life.

### SUMMARY

This application relates to compounds, compositions, and methods for determining the distribution and/or concentrations of a biomolecule (e.g., antisense oligonucleotides) in a subject.

This disclosure provides compounds and compositions that are useful in evaluating the distribution and kinetics of an antisense oligonucleotide in a subject. Also featured are methods for assessing the concentration of an antisense oligonucleotide in a desired region (e.g., brain, spinal cord, etc.) in a subject.

In one aspect, the disclosure provides an antisense oligonucleotide linked to a trans-cyclooctene.

In some instances, the antisense oligonucleotide is a CNS-penetrant ASO (i.e., an ASO that crosses the blood brain barrier). In some instances, the antisense oligonucleotide targets a target found in cells of the brain and/or spinal cord. In certain instances, the trans-cyclooctene is directly linked to the antisense oligonucleotide. In other instances, the trans-cyclooctene is linked to the antisense oligonucleotide via a linker. In some cases, the linker is an alkylene linker. In some instances, the trans-cyclooctene is linked to the antisense oligonucleotide at the 5′-end of the antisense oligonucleotide. In some instances, the trans-cyclooctene is linked to the antisense oligonucleotide at the 3′-end of the antisense oligonucleotide. In some instances, the antisense oligonucleotide is linked to the linker via a phosphorothioate linkage. In some instances, the antisense oligonucleotide (ASO) linked to the trans-cyclooctene has a structure of Formula I:

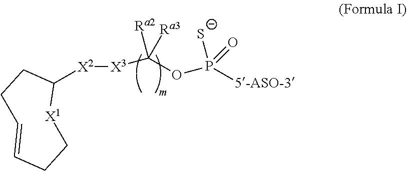

or a salt thereof, wherein X¹, X², X³, R^{a1}, R^{a2}, R^{a3} and m are as described herein and 5′-ASO-3′ is an antisense oligonucleotide.

In some instances, the antisense oligonucleotide (ASO) linked to the trans-cyclooctene has a structure of Formula I′:

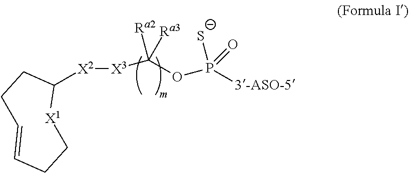

or a salt thereof, wherein X¹, X², X³, R^{a1}, R^{a2}, R^{a3} and m are as described herein and 3′-ASO-5′ is an antisense oligonucleotide.

In another aspect, the disclosure features a compound of Formula II:

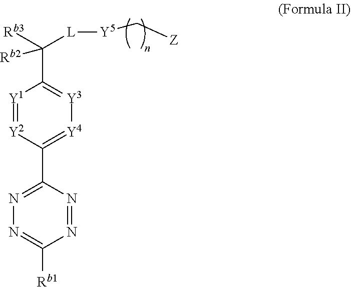

or a salt thereof, wherein Z, Y¹, Y², Y³, Y⁴, L, Y⁵, R^{b1}, R^{b2}, R^{b3}, R^{b4}, R^{b5}, and n are as defined herein.

In some instances, the disclosure provides a process of preparing a compound disclosed herein. In some instances, provided herein is a process of preparing an antisense oligonucleotide (ASO) linked to the trans-cyclooctene having a structure of Formula I. In some instances, provided herein is a process of preparing an antisense oligonucleotide (ASO) linked to the trans-cyclooctene having a structure of Formula I′. In some instances, provided herein is a process of preparing a compound of Formula II.

In another aspect, the disclosure relates to a method of determining the distribution of a biomolecule in a subject. The method involves administering the biomolecule linked to a trans-cyclooctene to the subject, followed by administering a radiolabeled tetrazine to the subject. The method further includes imaging the distribution of the biomolecule in the subject.

In another aspect, the disclosure relates to a method of determining the distribution of an antisense oligonucleotide in a subject. The method involves administering an antisense oligonucleotide linked to a trans-cyclooctene to the subject, followed by administering a radiolabeled tetrazine to the subject. The method further includes imaging the distribution of the antisense oligonucleotide in the subject.

In another aspect, the disclosure features a method of determining the distribution of an antisense oligonucleotide in the brain and/or spinal cord of a subject. The method involves administering an antisense oligonucleotide linked to a trans-cyclooctene to the subject. Next, the subject is administered a central nervous system penetrant radiolabeled tetrazine. This is followed by imaging the distribution of the antisense oligonucleotide in the brain and/or spinal cord of the subject.

In another aspect, the disclosure features a method of determining the concentration of a biomolecule in a desired location in a subject. The method involves administering a biomolecule linked to a trans-cyclooctene to the subject. Next, the subject is administered a radiolabeled tetrazine. Then, the concentration of the antisense oligonucleotide in the desired location of the subject is assessed.

In another aspect, the disclosure features a method of determining the concentration of an antisense oligonucleotide in the brain and/or spinal cord of a subject. The method involves administering an antisense oligonucleotide linked to a trans-cyclooctene to the subject. Next, the subject is administered a central nervous system penetrant radiolabeled tetrazine. Then, the concentration of the antisense oligonucleotide in the brain and/or spinal cord of the subject is determined.

In some instances, of the above methods, the tetrazine is radiolabeled with a radiolabel selected from the group consisting of fluorine-18, carbon-11, and gallium-68. In one instance, the tetrazine is radiolabeled with fluorine-18. In some instances, the tetrazine is a compound disclosed herein. In some instances, the tetrazine is Compound 1. In certain instances, the antisense oligonucleotide linked to trans-cyclooctene is one of those disclosed herein. In some instances, the antisense oligonucleotide linked to trans-cyclooctene is administered by intrathecal injection. In some instances, the radiolabeled tetrazine is administered by intravenous injection. In certain instances, the antisense oligonucleotide linked to trans-cyclooctene is administered by intrathecal injection and the radiolabeled tetrazine is administered by intravenous injection. In some cases, the radiolabeled tetrazine is administered about 24 hours after the administration of the antisense oligonucleotide linked to trans-cyclooctene. In some instances, the imaging is performed by PET. In certain instances, the imaging is performed by PET/CT. In other instances, the imaging is performed by SPECT. In yet other instances, the imaging is performed by SPECT/CT. In certain instances, the subject is a human.

In another aspect, the disclosure provides a pharmaceutical composition comprising a biomolecule linked directly or indirectly to a TCO described herein, and a pharmaceutically acceptable carrier. In some instances, the biomolecule is an antibody (a monovalent whole antibody, a bispecific whole antibody), an antigen-binding fragment (Fab, Fab′, F(ab)2, scFv, sc(Fv)2, diabody, nanobody), a peptide, or a nucleic acid (e.g., an antisense oligonucleotide). In some instances, the pharmaceutically acceptable carrier is phosphate buffered saline. In some instances, the pharmaceutically acceptable carrier is a-CSF. In some instances, the pharmaceutically acceptable carrier is sterile water for injection.

In another aspect, the disclosure provides a pharmaceutical composition comprising a radiolabeled tetrazine compound described herein; and a pharmaceutically acceptable carrier. In some instances, the tetrazine compound is labeled with fluorine-18. In certain cases, the tetrazine compound is CNS-penetrant (i.e., it can cross the blood brain barrier). In certain instances, the tetrazine compound is one of the tetrazine compounds disclosed herein. In one case, the tetrazine compound is Compound 1. In some instances, the pharmaceutically acceptable carrier is phosphate buffered saline. In some instances, the pharmaceutically acceptable carrier is a-CSF. In some instances, the pharmaceutically acceptable carrier is sterile water for injection.

In another aspect, the disclosure provides a composition comprising a biomolecule linked directly or indirectly to a TCO; and a radiolabeled tetrazine compound. In some instances, the tetrazine compound is labeled with fluorine-18. In certain cases, the tetrazine compound is CNS-penetrant (i.e., it can cross the blood brain barrier). In certain instances, the tetrazine compound is one of the tetrazine compounds disclosed herein. In one case, the tetrazine compound is Compound 1. In some instances, the biomolecule is an antibody (a monovalent whole antibody, a bispecific whole antibody), an antigen-binding fragment (Fab, Fab′, F(ab)₂, scFv, sc(Fv)₂, diabody, nanobody), a peptide, or a nucleic acid (e.g., an antisense oligonucleotide). In some cases, the composition is a pharmaceutical composition.

In another aspect, provided is a kit comprising a biomolecule linked directly or indirectly to a TCO; and a radiolabeled tetrazine compound. In some instances, the biomolecule is an antibody (a monovalent whole antibody, a bispecific whole antibody), an antigen-binding fragment (Fab, Fab′, F(ab)2, scFv, sc(Fv)2, diabody, nanobody), a peptide, or a nucleic acid (e.g., an antisense oligonucleotide). In one case, the biomolecule is an antisense oligonucleotide. In one case, the radiolabeled tetrazine compound is Compound 1. In some instances, the composition further comprises one or more of PBS, a-CSF, and sterile water for injection. In some cases, the kit comprises an injection device. In some cases, included is an injection device for intrathecal administration. In some cases, included is an injection device for intravenous administration. In some cases, included are an injection device for intrathecal administration and an injection device for intravenous administration.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF THE DRAWINGS

FIG. 1 shows the inverse electron-demand Diels-Alder [4+2] (IEDDA) cycloaddition click ligation between a 1,2,4,5 tetrazine and trans-cyclooctene that is utilized herein to link the radioligand to the ASO in vivo. This is an exceptionally fast reaction (k₂>30,000 M⁻¹s⁻¹) that is also bioorthogonal.

FIG. 2 shows confocal images of HeLa cells incubated with either a Malat1 ASO, Malat1 ASO-TCO, or Malat1 ASO-PEG4-TCO for 24 h at 37° C. and then fixed, permeabilized, and stained using tetrazine-Cy5.

FIG. 3 is a schematic representation of an illustrative example of pretargeting. First, an ASO-TCO is injected intrathecally, where it distributes through the CSF and is internalized into the parenchyma of the brain and spine. Next, following a delay-period of hours or days, the ¹⁸F-537-Tz radiotracer is injected intravenously where it is carried throughout the body and enters into the central nervous system (CNS) through the blood-brain-barrier. The tracer binds covalently to any ASO-TCO it encounters and localizes in tissue, while unbound tracer is cleared. Remaining signal by PET can therefore be attributed to ASO-TCO in tissue that has reacted with the radiotracer.

FIG. 4 are PET/CT images showing specific uptake of tracer in the brain and spinal cord in rats treated with ASO-TCO. Sprague-Dawley rats were administered ASO-TCO intrathecally (2.5 mM in 30 μL saline). After either 24 hours (center) or 168 hours (right), those rats were then injected I.V. with ¹⁸F-537-Tz (22-30 MBq; 148 MBq/μmol; 0.2 nmol). Rats were imaged by PET-CT from 75-90 min. post-injection of radiotracer. Control rats (left) received saline only.

FIG. 5 is a bar graph showing Regions of Interest (ROI) drawn from the PET data. Ratios were obtained of % ID/g in tissue of interest (brain and spine) to the reference region (heart). Significant differences were observed between rats that received ASO-TCO compared to those that did not. Lanes in bar graph depicted from left to right: ASO-TCO (brain:heart); Naive (brain:heart); ASO-TCO (spine:heart); and Naive (spine:heart).

FIG. 6 provides the results of autoradiography of brains resected following imaging and shows a distinct pattern of distribution that matches that of ASO in the brain.

FIG. 7 shows an exemplary analytical HPLC of Compound 1.

FIG. 8 shows an exemplary analytical HPLC of co-injection of Compound 1 with the reference standard (Compound 1′).

FIG. 9 depicts Time Activity Curves (TACs) showing baseline (left) and self-block (right) in the same rat. For the baseline graph, the curves are from top to bottom (top and bottom referring to the latest time point): hypothalamus, thalamus, cerebellum, cortex, striatum, brain, hippocampus, and frontal cortex. For the self-block graph, the curves are from top to bottom (top and bottom referring to the latest time point): thalamus, hypothalamus, striatum, cortex, brain, hippocampus, cerebellum, and frontal cortex.

FIG. 10 provides representative PET/CT images (coronal) showing significantly higher uptake in rats treated with Malat1 ASO-TCO (bottom) over baseline (top).

FIG. 11 is a TAC comparing baseline (n=3) and pretargeted (n=3) cohorts.

FIG. 12 shows ROI delineation of the spinal cord and CSF.

FIG. 13 depicts parent fraction analysis of blood plasma and blood SUV values. Tracer behavior in the blood was consistent between scans.

FIG. 14 shows PET scans for baseline, post-ASO dosing at 24 h and 168 h.

FIG. 15 shows baseline time-activity curves for brain subregions following i.v. injection of ¹⁸F-537-Tz.

FIG. 16 illustrates whole-brain TAC for each scan (left); and scan TACs normalized to their maximum showing differences in clearance rate between scans (right).

FIG. 17 depicts a static scan showing ROI drawn over spinal cord (left); and SUV to muscle ratio in scans (right).

### DETAILED DESCRIPTION

This disclosure relates in part to compounds and compositions that are useful for “pretargeting.” “Pretargeting” separates the delivery of a radiolabelled compound/radioligand from that of a modified biomolecule or vector (e.g. AAV, nanoparticles) or targeting agent (e.g., ASO). These compounds and compositions can be used in vivo, e.g., to evaluate distribution and/or concentration of a biomolecule (e.g., antisense oligonucleotide (ASO)) in a subject. In some cases, the compounds and compositions are used to evaluate the distribution and/or concentration of a biomolecule (e.g., ASO) in the brain and/or spinal cord of a subject. The working examples describe in vivo the covalent binding of a radiolabeled compound or radioligand (e.g., radiolabeled tetrazine) and a targeting agent (e.g., ASO-TCO) via an inverse electron demand Diels-Alder (IEDDA) reaction for pretargeting in the brain, as well as for pretargeted imaging for measuring ASO distribution. As current ASO imaging relies on direct labeling with longer-lived radioisotopes, the compounds, compositions and methods disclosed herein significantly impact the development of new ASO-based therapies by elucidating long-term temporal distributions in vivo while maintaining a low radiation exposure to the patient.

### Pretargeting

Medical diagnosis and therapy routinely makes use of imaging agents. Such agents can be useful, e.g., to determine if a therapeutic agent has reached its intended target and to determine the location and/or concentration of a therapeutic or diagnostic agent. Existing methods can however be problematic. For example, the relatively slow pharmacokinetics of certain biomolecules used for imaging require the attached radioactive label to have multiday half-lives because if distribution of the biomolecules is to be assessed at longer time-points there must remain sufficient radiation to image successfully. In some instances, this leads to high activity concentrations in and radiation doses to non-target organs. To circumvent these problems an alternative approach has emerged, that is referred to as “pretargeting” whereby a radiolabeled compound or radioligand (e.g., radiolabeled tetrazine) and a modified biomolecule or vector (e.g. AAV, nanoparticles) or targeting agent (e.g., ASO-TCO) are delivered separately to a subject.

Pretargeted methods generally involve the following steps: first, the injection into the subject of a modified biomolecule or vector (e.g. AAV, nanoparticles) or targeting agent (e.g., ASO) that binds or localizes to the target of interest but also has the ability to bind to a radioligand; second, the slow accumulation of the modified biomolecule or vector (e.g. AAV, nanoparticles) or targeting agent (e.g., ASO) at the site of the target and concomitant clearance of the modified biomolecule or vector (e.g. AAV, nanoparticles) or targeting agent from the blood; third, the injection into the bloodstream of the radiolabeled compound or radioligand (e.g., small-molecule radioligand); and fourth the binding of the radiolabeled compound or radioligand to the modified biomolecule or vector (e.g. AAV, nanoparticles) or targeting agent (e.g., ASO), followed by the rapid clearance of excess radioactivity. In some instances, an additional step is added before the injection of the radiolabeled compound or radioligand, specifically, the administration of a clearing agent designed to accelerate the removal of residual targeting agent from the bloodstream. In another aspect, the pharmacokinetics of the radiolabeled compound or radioligand not only reduces background radiation dose to non-target organs but also facilitates the use of radioisotopes with short half-lives that would normally be incompatible with such imaging.

Pretargeting approaches have been used for targeting agents such as antibodies and peptides that are not internalized upon binding their target. This disclosure, in striking contrast, applies pretargeting to internalized targeting agents such as ASOs. In addition, this disclosure provides compounds and compositions that can be used for penetrating the blood brain barrier and thus having utility in pretargeting in the central nervous system.

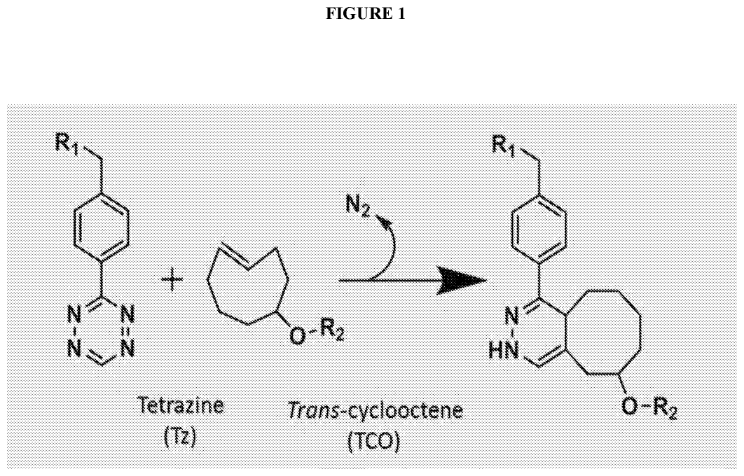

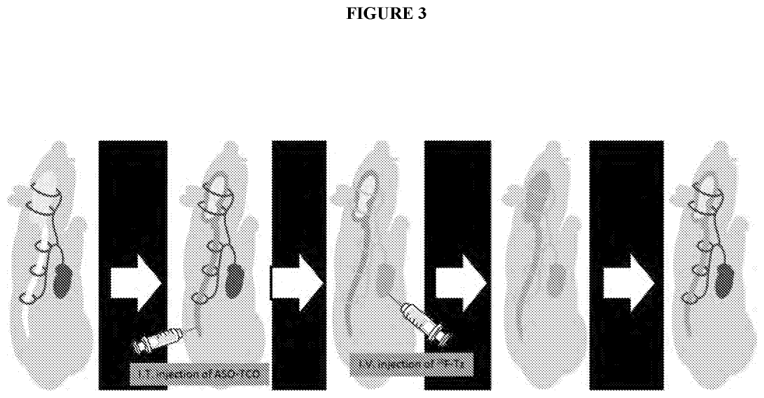

An illustrative example of applying pretargeting in the central nervous system is depicted in FIG. 3. As can be seen in this example, pretargeting separates the delivery of the radioactivity from the targeting agent (e.g., ASO). The targeting agent (e.g., ASO) is modified with a trans-cyclooctene (TCO) and is injected intrathecally while the radioligand contains a 1,2,4,5 tetrazine (Tz) and is injected intravenously. The Tz and TCO undergo an inverse electron demand Diels-Alder (IEDDA) reaction in vivo, covalently binding the radioligand to the ASO (FIG. 1).

### Antisense Oligonucleotide (ASO)-Trans-Cyclooctene (TCO) Fusions (“ASO-TCO”)

Provided herein is an antisense oligonucleotide (ASO) linked to a trans-cyclooctene (“ASO-TCO”). The trans-cyclooctene can be directly linked to the ASO. In some instances, the trans-cyclooctene is linked to the ASO via a linker, such as an alkylene linker. The trans-cyclooctene can be either directly or indirectly linked to the ASO at the 5′-end of the ASO. The trans-cyclooctene can also be either directly or indirectly linked to the ASO at the 3′-end of the ASO. In some instances, the ASO is linked to the linker (as defined herein) via a phosphorothioate linkage. In some cases, the trans-cyclooctene is a trans-cyclooctene with a cis-ring fusion with cyclopropane (“s-TCO”). In some cases, the trans-cyclooctene is a trans-cyclooctene fused with a dioxolane ring (“d-TCO”).

In some aspects, the modified biomolecule or targeting agent is an antisense oligo nucleotide (“ASO”). According to the invention, the ASO can be any ASO know in the art. ASOs are synthetic single stranded strings of nucleic acids that bind to ribonucleic acid (RNA) and thereby alter or reduce expression of the target RNA. They can not only reduce expression of proteins by breakdown of the targeted transcript, but also restore protein expression or modify proteins through interference with pre-mRNA splicing. This disclosure encompasses ASOs of both types. In certain instances, the ASO of this disclosure is a “gapmer.” Such ASOs primarily act by selectively cleaving mRNAs that have complementary sites through an RNase H-dependent mechanism. They have a central region that supports RNase H activity flanked by chemically modified ends that increase affinity and/or reduce susceptibility to nucleases. In some instances, the ASO of this disclosure is a splice switching oligonucleotide (SSO) (e.g., nusinersen). SSOs are generally fully modified so as to ablate RNase H activity and allow interaction with nuclear pre-mRNA during splicing. They can be designed to bind to the 5′ or 3′ splice junctions or to exonic splicing enhancer or silencer sites. By binding to such sites they can modify splicing by, e.g., promoting alternative use of exons, exon exclusion, or exon inclusion.

A non-limiting example of an ASO that is encompassed by this disclosure is provided below.

| | Antisense Oligonucleotide | | |
|---|---|---|---|
| Name | Sequence | Design | Length |
| MALAT1 | G^{Me}Co^{Me}CoAoGG^{Me}CTGGTTATG | control | 20 |
| | Ao^{Me}Co^{Me}U^{Me}CA | | |
| | (SEQ ID NO: 1) | | |

wherein: the underlined nucleoside has a 2′-O-(2-methoxyethyl) (MOE) modification;
the “o” is a phosphodiester internucleoside linkage and the absence of “o” indicates a phosphorotioate internucleoside linkage;
“^{Me}U” is 5-methyl-uracil; and
“^{Me}C” is 5-methyl-cytosine.

In some instances, the antisense oligonucleotide is a gapmer or a splice switching antisense oligonucleotide. In some cases, the antisense oligonucleotide consists of 12 to 20 nucleosides (e.g., 12, 13, 14, 15, 16, 17, 18, 19, 20). In some instances, the antisense oligonucleotide is one that needs to cross the blood brain barrier. In some instances, the antisense oligonucleotide is one that is useful for the treatment of a neurodegenerative disorder. In some instances, the antisense oligonucleotide is one that is useful for the treatment of any one of: spinal muscular atrophy; amyotrophic lateral sclerosis, Alzheimer's disease, Parkinson's disease (familial or sporadic), frontotemporal dementia, myotonic dystrophy type 1, Huntington's disease, Angelman syndrome, Creutzfeldt-Jakob disease, Spinocerebellar Ataxia Type 3, and Menkes disease.

In some instances, the antisense oligonucleotide linked to the trans-cyclooctene has a structure of Formula I:

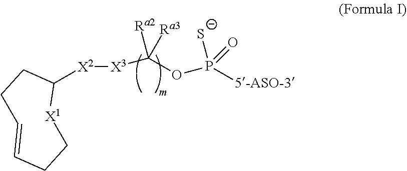

or a salt thereof, wherein:

X¹ is CH₂ or O;

X² is (CH₂)ₜ;

X³ is O, C(O), C(O)O, OC(O), or OC(O)NR^{a1};

R^{a1} is H, C₁₋₆ alkyl, or C₁₋₆ haloalkyl;

R^{a2} and R^{a3} are each independently H or C₁₋₆ alkyl;

t is 0 or 1;

m is 1, 2, 3, 4, 5, 6, 7, 8, 9, or 10; and

5′-ASO-3′ is an antisense oligonucleotide.

In some instances, the antisense oligonucleotide linked to the trans-cyclooctene has a structure of Formula I′:

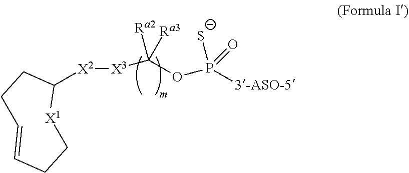

or a salt thereof, wherein:

X¹ is CH₂ or O;

X² is (CH₂)ₜ;

X³ is O, C(O), C(O)O, OC(O), or OC(O)NR^{a1};

R^{a1} is H, C₁₋₆ alkyl, or C₁₋₆ haloalkyl;

R^{a2} and R^{a3} are each independently H or C₁₋₆ alkyl;

t is 0 or 1;

m is 1, 2, 3, 4, 5, 6, 7, 8, 9, or 10; and

3′-ASO-5′ is an antisense oligonucleotide.

The moiety

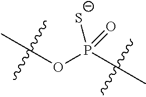 [SMILES: *OP(*)(=O)[S-]]

may exist as an anion as depicted, where the charge is balanced by a suitable cation such as Na⁺, K⁺, and the like. In some instances, the cation is Na⁺. Depending on the pH environment, the charge may be balanced by a proton.

The trans-cyclooctene compound provided herein include a compound that is not linked to the biomolecule (e.g., ASO), e.g.,

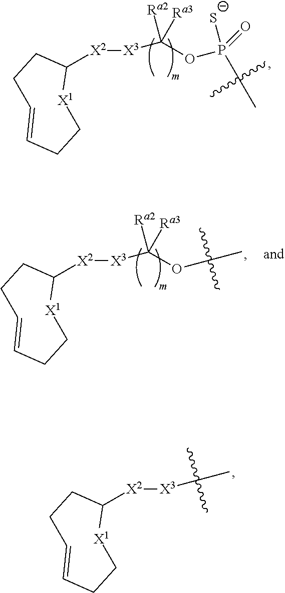

which may be synthesized or purchased from a commercial source, and the wavy line denotes a point of attachment to a group that is suitable to be coupled with a biomolecule such as ASO. Commercially available trans-cyclooctene compounds include TCO-PNB (Click Chem Tools: 1192) and TCO-NHS (Click Chem Tools: 1016).

The trans-cyclooctene compounds described herein can be linked directly or indirectly to a biomolecule. Exemplary biomolecules include antisense oligonucleotides, antibodies, antigen-binding antibody fragments, peptides, and small molecules.

In some instances, the antisense oligonucleotide linked to the trans-cyclooctene has a structure of Formula Ia:

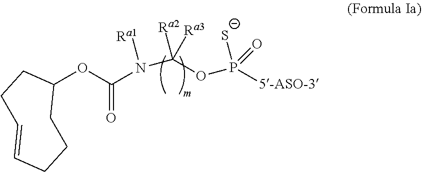

or a salt thereof, wherein R^{a1}, R^{a2}, R^{a3} and m are as defined herein.

In some instances, the antisense oligonucleotide linked to the trans-cyclooctene has a structure of Formula I′a:

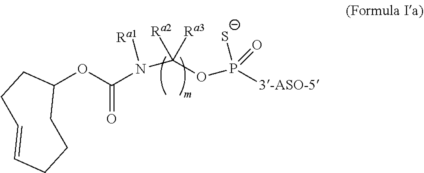

or a salt thereof, wherein R^{a1}, R^{a2}, R^{a3} and m are as defined herein.

In some instances, the antisense oligonucleotide linked to the trans-cyclooctene has a structure of Formula Ib:

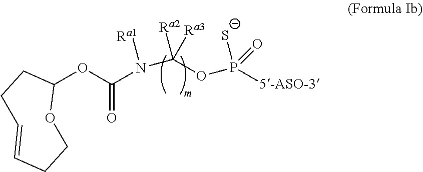

or a salt thereof, wherein R^{a1}, R^{a2}, R^{a3} and m are as defined herein.

In some instances, the antisense oligonucleotide linked to the trans-cyclooctene has a structure of Formula I′b:

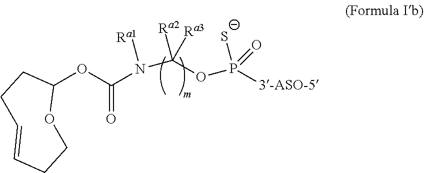

or a salt thereof, wherein R^{a1}, R^{a2}, R^{a3} and m are as defined herein.

In some instances, the antisense oligonucleotide linked to the trans-cyclooctene has a structure of Formula Ic:

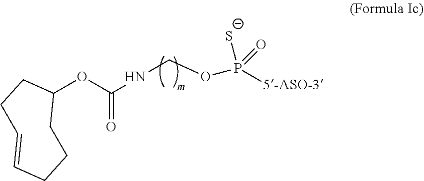

or a salt thereof, wherein m is as defined herein.

In some instances, the antisense oligonucleotide linked to the trans-cyclooctene has a structure of Formula I′c:

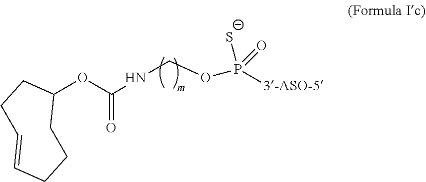

or a salt thereof, wherein m is as defined herein.

In some instances, the antisense oligonucleotide linked to the trans-cyclooctene has a structure of Formula Id:

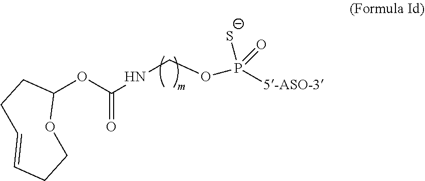

or a salt thereof, wherein m is as defined herein.

In some instances, the antisense oligonucleotide linked to the trans-cyclooctene has a structure of Formula I′d:

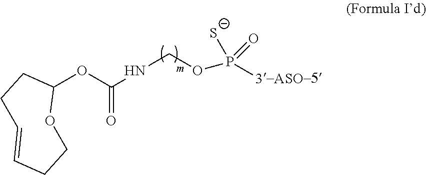

or a salt thereof, wherein m is as defined herein.

In some instances, X¹ is CH₂. In some instances, X¹ is O. In some instances, t is 0. In some instances, t is 1. In some instances, X³ is OC(O)NR^{a1} such as OC(O)NH. In some instances, X³ is C(O)O or OC(O). In some instances, X³ is O. In some instances, X³ is C(O). In some instances, R^{a1} is H. In some instances, R^{a1} is C₁₋₆ alkyl such as methyl, ethyl, propyl, and the like. In some instances, R^{a1} is C₁₋₆ haloalkyl such as trifluoromethyl, difluoromethyl, and the like. In some instances, R^{a2} is H. In some instances, R^{a2} is C₁₋₆ alkyl such as methyl, ethyl, propyl, etc. In some instances, R^{a3} is H. In some instances, R^{a3} is C₁₋₆ alkyl such as methyl, ethyl, propyl, etc. In some instances, R^{a2} and R^{a3} are H. In some instances, R^{a2} is H and R^{a3} is C₁₋₆ alkyl. In some instances, m is 1, 2, 3, 4, 5, or 6. In some instances, m is 1. In some instances, m is 2. In some instances, m is 3. In some instances, m is 4. In some instances, m is 5. In some instances, m is 6.

In some instances, the antisense oligonucleotide linked to the trans-cyclooctene has the following structure:

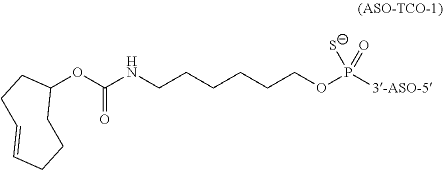

or a salt thereof.

In some instances, the antisense oligonucleotide linked to the trans-cyclooctene has the following structure:

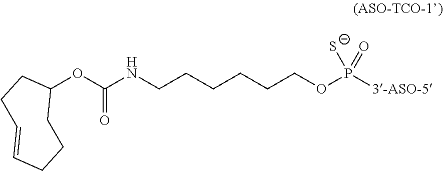

or a salt thereof.

The modified biomolecule or vector or targeting agent described herein may be administered to a subject by any suitable means known in the art (e.g., intrathecally, intravenously, intracranially, etc). The modified biomolecule or vector or targeting agent described herein may be administered to a subject intrathecally. The modified biomolecule or vector or targeting agent described herein may be administered to a subject intravenously. The modified biomolecule or vector or targeting agent described herein may be administered to a subject intracranially.

### Tetrazine Compounds

In some aspects, the radiolabeled molecule or radioligand is a radiolabeled tetrazine compound as described herein. Provided herein are tetrazine compounds suitable for in vivo inverse electron demand Diels-Alder reaction with a modified biomolecule/targeting vector (e.g. ASO-TCO) as described herein. The radiolabel of the radiolabeled molecule/radioligand may be any radioactive isotope suitable for diagnostic imaging (as described below) known in the art. In some aspects, the radioactive isotope is ¹⁸F, ¹¹C, or ⁶⁸Ga. In some aspects, the radioactive isotope is ¹⁸F. In some aspects, the radioactive isotope is ¹¹C. In some aspects, the radioactive isotope is ⁶⁸Ga.

In some instances, provided herein is a compound of Formula II:

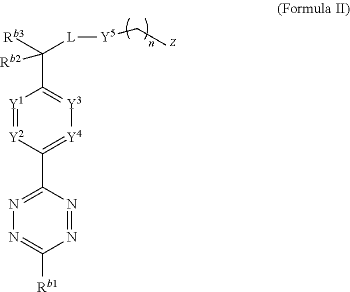

or a salt thereof, wherein

Z is ¹⁸F, ¹¹C-moiety, or chelated ⁶⁸Ga;

Y¹, Y², Y³, and Y⁴ are each independently CH or N;

L is C(O)NR^{b4}, NR^{b4}C(O), O, OCH₂, NR^{b5}, or NR^{b5}CH₂;

Y⁵ is a bond, 5-6 membered heteroaryl or phenyl;

R^{b1} is H, C₁₋₆ alkyl, C₁₋₆ haloalkyl, pyridinyl, or pyrimidinyl;

R^{b2}, R^{b3}, R^{b4}, and R^{b5} are each independently H or C₁₋₆ alkyl; and

n is 0, 1, 2, 3, 4, 5, 6, 7, 8, 9 or 10.

In some instances, provided herein is a compound of Formula IIa:

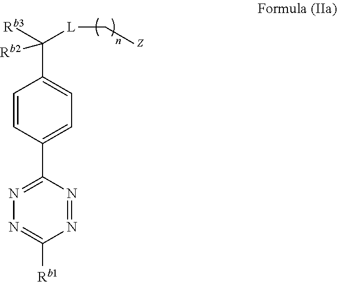

or a salt thereof, wherein Z, R^{b1}, R^{b2}, R^{b3}, L, and n are as defined herein.

In some instances, provided herein is a compound of Formula IIb:

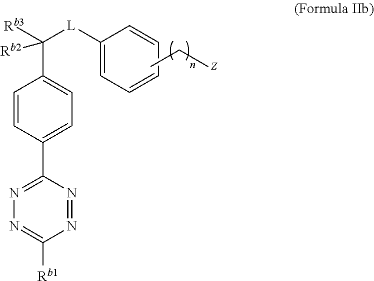

or a salt thereof, wherein Z, R^{b1}, R^{b2}, R^{b3}, L, and n are as defined herein.

In some instances, provided herein is a compound of Formula IIc:

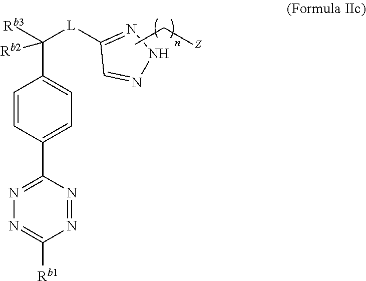

or a salt thereof, wherein Z, R^{b1}, R^{b2}, R^{b3}, L, and n are as defined herein.

In some instances, provided herein is a compound of Formula IId:

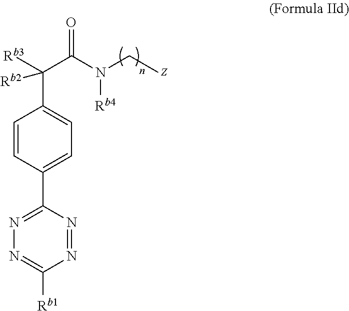

or a salt thereof, wherein Z, R^{b1}, R^{b2}, R^{b3}, R^{b4}, and n are as defined herein.

In some instances, provided herein is a compound of Formula IIe:

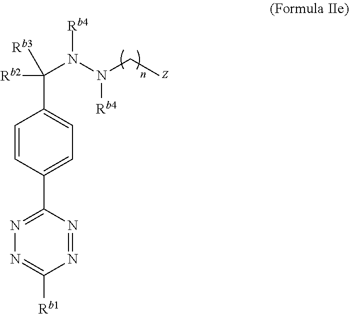

or a salt thereof, wherein Z, R^{b1}, R^{b2}, R^{b3}, R^{b4}, and n are as defined herein.

In some instances, provided herein is a compound of Formula IIf:

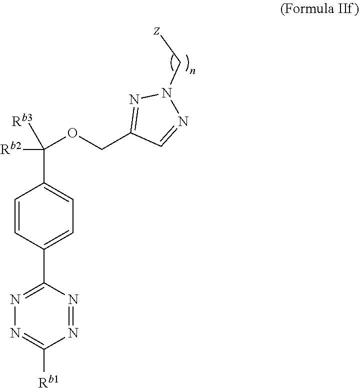

or a salt thereof, wherein Z, R^{b1}, R^{b2}, R^{b3}, and n are as defined herein.

In some instances, provided herein is a compound of Formula IIg:

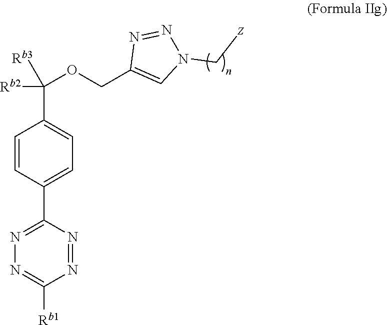

or a salt thereof, wherein Z, R^{b1}, R^{b2}, R^{b3}, and n are as defined herein.

In some instances, Z is ¹⁸F. In some instances, Z is an ¹¹C-moiety, e.g., ¹¹CH₃, ¹¹CH₂—CH₃, ¹¹CH₂—CH₂—CH₃, and the like. In some instances, Z is chelated ⁶⁸Ga.

In some instances, Y⁵ is a bond. In some instances, Y⁵ is 5-6 membered heteroaryl. In some instances, Y⁵ is 5-membered heteroaryl such as triazolyl, imidazolyl, or pyrazolyl. In some instances, Y⁵ is triazolyl. In some instances, Y⁵ is phenyl.

In some instances, L is C(O)NR^{b4} such as C(O)NH and C(O)N(CH₃). In some instances, L is NR^{b4}C(O) such as NH(CO) and N(CH₃)C(O). In some instances, L is O. In some instances, L is OCH₂. In some instances, L is NR^{b5}CH₂ such as NHCH₂ and N(CH₃)CH₂.

In some instances, R^{b1} is H or C₁₋₆ alkyl. In some instances, R^{b1} is H. In some instances, R^{b1} is C₁₋₆ alkyl such as methyl, ethyl, propyl, and the like. In some instances, R^{b1} is C₁₋₆ haloalkyl such as trifluoromethyl, difluoromethyl, and the like. In some instances, R^{b1} is pyridinyl. In some instances, R^{b1} is pyrimidinyl.

In some instances, R^{b2} is H. In some instances, R^{b2} is C₁₋₆ alkyl such as methyl, ethyl, propyl, and the like. In some instances, R^{b3} is H. In some instances, R^{b3} is C₁₋₆ alkyl such as methyl, ethyl, propyl, and the like. In some instances, R^{b2} and R^{b3} are H. In some instances, R^{b2} is H and R^{b3} is C₁₋₆ alkyl such as methyl, ethyl, propyl, and the like.

In some instances, R^{b4} is H. In some instances, R^{b4} is C₁₋₆ alkyl such as methyl, ethyl, propyl, and the like. In some instances, R^{b5} is H. In some instances, R^{b5} is C₁₋₆ alkyl such as methyl, ethyl, propyl, and the like.

In some instances, n is 0, 1, 2, 3, or 4. In some instances, n is 0. In some instances, n is 1. In some instances, n is 2. In some instances, n is 3. In some instances, n is 4.

In some instances, provided herein is a compound is selected from:

| Compound no | Structure |
|---|---|
| 1 | 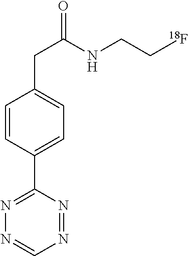 [SMILES: O=C(Cc1ccc(-c2nncnn2)cc1)NCC[18F]] |
| 2 | 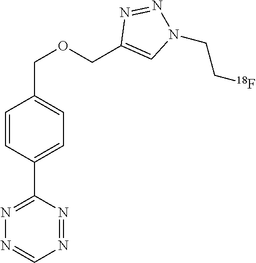 [SMILES: [18F]CCn1cc(COCc2ccc(-c3nncnn3)cc2)nn1] |
| 3 | 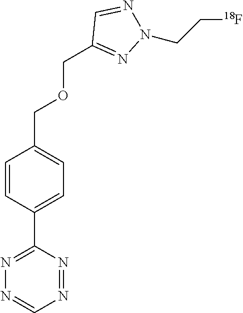 [SMILES: [18F]CCn1ncc(COCc2ccc(-c3nncnn3)cc2)n1] |
| 4 | 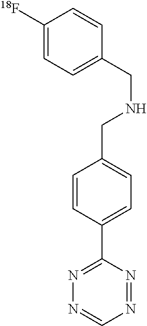 [SMILES: [18F]c1ccc(CNCc2ccc(-c3nncnn3)cc2)cc1] |
| 5 | 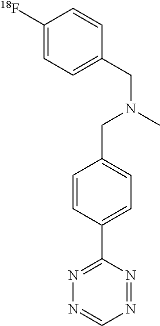 [SMILES: CN(Cc1ccc([18F])cc1)Cc1ccc(-c2nncnn2)cc1] |
| 6 | 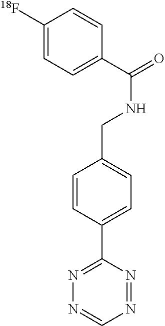 [SMILES: O=C(NCc1ccc(-c2nncnn2)cc1)c1ccc([18F])cc1] |
| 7 | 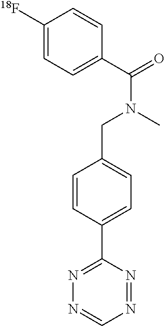 [SMILES: CN(Cc1ccc(-c2nncnn2)cc1)C(=O)c1ccc([18F])cc1] |
| | and |
| 8 | 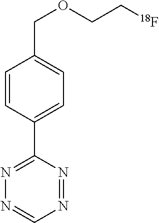 [SMILES: [18F]CCOCc1ccc(-c2nncnn2)cc1] |

or a salt thereof.

In some instances, Compound 1 is also referred to in this disclosure as ¹⁸F-537-Tz.

The radiolabeled molecule or radioligand described herein may be administered to a subject by any suitable means known in the art (e.g., intrathecally, intravenously, intracranially, etc). The radiolabeled molecule or radioligand described herein may be administered to a subject intrathecally. The radiolabeled molecule or radioligand described herein may be administered to a subject intravenously. The radiolabeled molecule or radioligand described herein may be administered to a subject intracranially.

Provided herein are also the corresponding non-radiolabeled tetrazine compounds and process of preparing the same. For example, provided herein are compounds selected from:

| Compound no | Structure |
|---|---|
| 1′ | 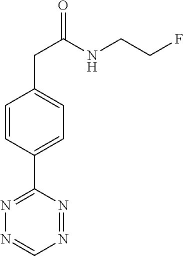 [SMILES: O=C(Cc1ccc(-c2nncnn2)cc1)NCCF] |
| 2′ | 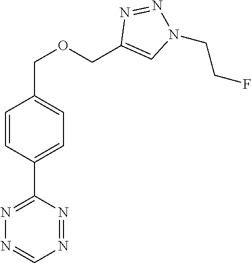 [SMILES: FCCn1cc(COCc2ccc(-c3nncnn3)cc2)nn1] |
| 3′ | 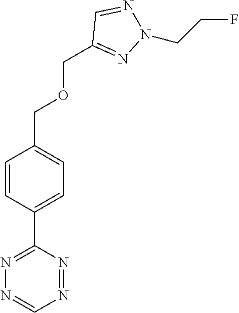 [SMILES: FCCn1ncc(COCc2ccc(-c3nncnn3)cc2)n1] |
| 4′ | 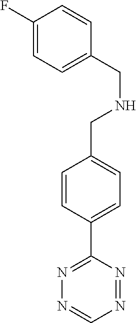 [SMILES: Fc1ccc(CNCc2ccc(-c3nncnn3)cc2)cc1] |
| 5′ | 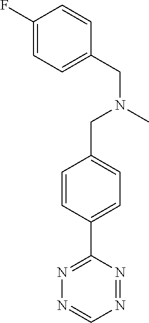 [SMILES: CN(Cc1ccc(F)cc1)Cc1ccc(-c2nncnn2)cc1] |
| 6′ | 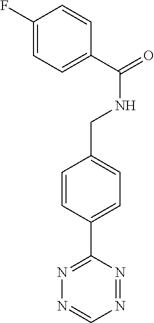 [SMILES: O=C(NCc1ccc(-c2nncnn2)cc1)c1ccc(F)cc1] |
| 7′ | 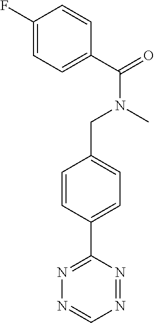 [SMILES: CN(Cc1ccc(-c2nncnn2)cc1)C(=O)c1ccc(F)cc1] |
| | and |
| 8′ | 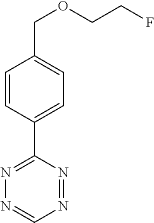 [SMILES: FCCOCc1ccc(-c2nncnn2)cc1] |

or a salt thereof.

Provided herein are also intermediate compounds prepared in the synthesis of the compounds described herein. In some instances, the intermediate compound is selected from:

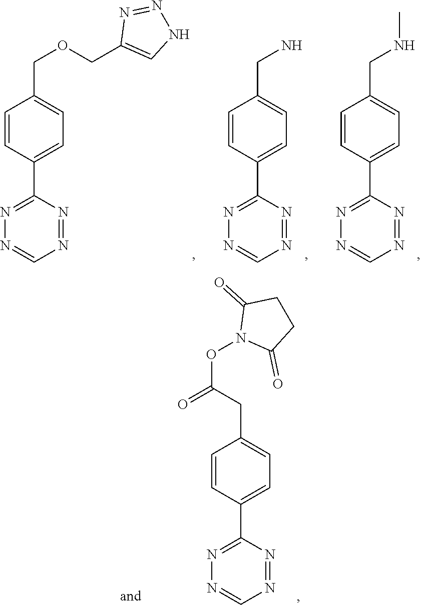

or a salt thereof.

### Methods of Preparing Compounds

Compounds provided herein and their salts thereof can be prepared using known synthesis techniques and can be synthesized according to any of numerous possible synthetic routes, such as those described herein.

The reactions for preparing compounds provided herein can be carried out in suitable solvents. Suitable solvents can be substantially nonreactive with the starting materials (reactants), the intermediates, or products at the temperatures at which the reactions are carried out. A given reaction can be carried out in one solvent or a mixture of more than one solvent.

Preparation of compounds provided herein can involve the protection and deprotection of various chemical groups. The chemistry of protecting groups can be found, for example, in T.W. Greene and P.G.M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), mass spectrometry, or by chromatography such as high performance liquid chromatography (HPLC) or thin layer chromatography (TLC).

In some instances, the disclosure provides a process of preparing a compound disclosed herein. In some instances, provided herein is a process of preparing a compound of Formula II. A compound of Formula II can be prepared according to Scheme A. For example, a compound of Formula I-1 can be converted to a tetrazine compound of Formula II using suitable agents. Agents suitable for such conversion includes catalyst such as Zn(OTf)₂ and Ni(OTf)₂, methanimidamide acetate, and NH₂NH₂. The conversion of a compound of Formula I-1 to a tetrazine compound of Formula II can further include NaNO₂ and HCl.

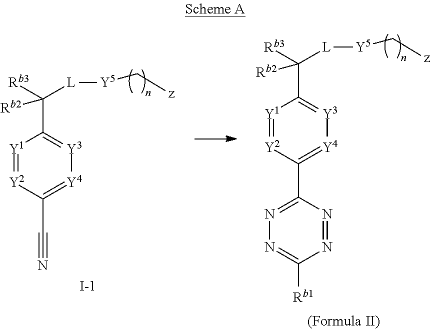

For example, Compound 1 can be prepared according to Scheme B below.

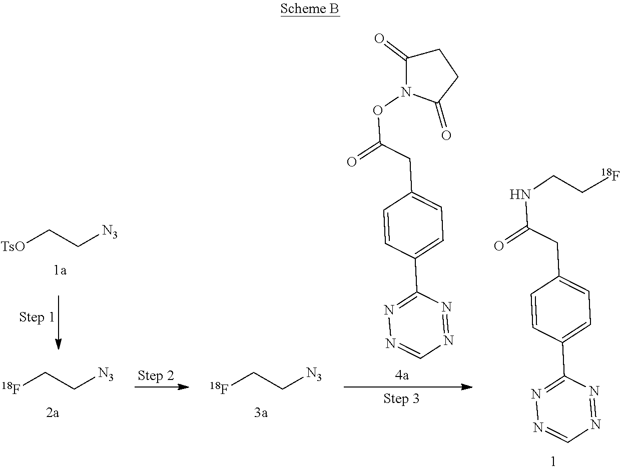 [SMILES: [N-]=[N+]=NCC[18F] ; Cc1ccc(S(=O)(=O)OCCN=[N+]=[N-])cc1 ; [N-]=[N+]=NCC[18F] ; O=C(Cc1ccc(-c2nncnn2)cc1)ON1C(=O)CCC1=O ; O=C(Cc1ccc(-c2nncnn2)cc1)NCC[18F]]

Compound 1 can be prepared by a process that comprises:

(1) converting

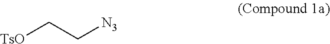 [SMILES: Cc1ccc(S(=O)(=O)OCCN=[N+]=[N-])cc1]

in the presence of ¹⁸F⁻ and K₂₂₂/K₂CO₃ to provide

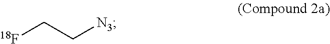

(2) reducing Compound 2a in the presence of a reducing agent to provide

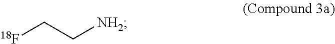

and
(3) reacting compound 3a with

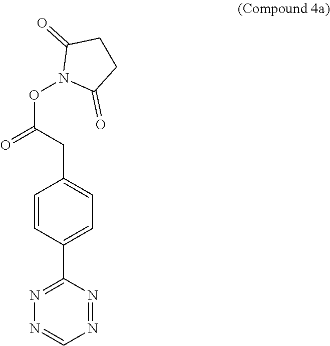 [SMILES: O=C(Cc1ccc(-c2nncnn2)cc1)ON1C(=O)CCC1=O]

to provide Compound 1.

In some instances, provided herein is a process of preparing Compound 1, wherein the process comprises reacting Compound 3a with Compound 4a. In some instances, the process of preparing Compound 1 includes preparing Compound 3a by a process comprising reducing Compound 2a in the presence of a reducing agent. In some instances, the process of preparing Compound 1 also includes preparing Compound 2a by a process comprising converting Compound 1a in the presence of ¹⁸F⁻ and K₂₂₂/K₂CO₃ to provide Compound 2a.

The process of converting Compound 1a to Compound 2a can be carried out in the presence of ¹⁸F⁻ and K₂₂₂/K₂CO₃. The converting can be carried out in a solvent such as an organic solvent like acetonitrile. The converting can be carried out at temperature of about 90° C. to about 120° C., e.g., about 100° C., about 105° C., and about 110° C.

The process of reducing Compound 2a to Compound 3a can be carried out in the presence of a reducing agent. For example, the reducing agent is Copper wire. The reducing of Compound 2a can be carried out in the presence of an acid such as trifluoroacetic acid in water. The reducing can be carried at temperature of about 60° C. to about 100° C., e.g., about 70° C., about 80° C., and about 90° C.

The process of reacting Compound 3a with Compound 4a to provide Compound 1 can be carried out in the presence of a base. For example, the base is an amine base such as N,N-diisopropylethylamine. The reacting can be carried out in an organic solvent such as dimethylformamide. The reducing can be carried at temperature of about 25° C. to about 50° C., e.g., about 30° C., about 37° C., and about 40° C.

In some instances, the preparation of the compounds provided herein can be carried out using GE Tracerlab. In some instances, the radiolabeled compounds provided herein can be made by means known in the art including but not limited to automated radiosynthesizers (e.g. TRACERlab FX2N.

In some instances, a trans-cyclooctene compound can be attached to the biomolecule (e.g., ASO at the 5′ position). In some instances, provided herein is a process of preparing an antisense oligonucleotide (ASO) linked to the trans-cyclooctene having a structure of Formula I. For example, a 5′-hexyl amino oligonucleotide (e.g., ASO) can be dissolved in a borate buffer and a TCO-PNB or TCO-NHS can be added to the buffer to generate a TCO linked oligonucleotide.

### Methods of Evaluating ASO Distribution

This disclosure features a method of evaluating the distribution of an ASO in a subject (e.g., a human). In some cases, the distribution of the ASO is assessed in the brain and/or spinal cord of the subject. The method involves administering to the subject an ASO linked to a trans-cyclooctene described herein. In some cases, the ASO linked to a trans-cyclooctene is administered intravenously. In certain cases, particularly where the distribution of the ASO is assessed in the brain and/or spinal cord, the ASO linked to a trans-cyclooctene is administered intrathecally. In certain cases, the ASO linked to a trans-cyclooctene is formulated in PBS or a-CSF. In some instances, the ASO limited to a trans-cyclooctene is a compound of Formula I, Ia, Ib, Ic, or Id, or Compound ASO-TCO-1 or Malat1 ASO-TCO. The method further involves administering a radiolabeled tetrazine compound disclosed herein to the subject. In some instances, the tetrazine is radiolabeled with any radionuclide or radioisotope for diagnostic imaging (as described herein) known in the art. In some instances, the tetrazine is radiolabeled with a radionuclide that decays exclusively or almost exclusively through positron emission. In some instances, the tetrazine is radiolabeled with a radionuclide that has a short half-life (less than 12 hours) or a moderate half-life (about 12 to 18 hours). In some instances, the tetrazine is radiolabeled with a fluorine-18, carbon-11, or gallium-68. In some instances, the tetrazine is radiolabeled with fluorine-18. In some instances, the tetrazine compound is a compound described, e.g., a compound of Formula II, IIa, IIb, IIc, IId, IIe, IIf, or IIg, or a compound selected from Compounds 1-7. In some instances, the radionuclide or radioisotope is covalently bonded to the radiolabeled compound/radioligand. In some instances, the radionuclide or radioisotope is bound to the radiolabeled compound/radioligand via a chelating moiety. The chelating moiety may be any suitable chelator known in the art (e.g., NOTA). In some cases, the radiolabeled tetrazine is administered intravenously. In certain cases, the radiolabeled tetrazine is formulated in PBS or a-CSF.

The timing of when the radiolabeled tetrazine is administered depends on the half-life of each of the ASO and the TCO. In some cases, the radiolabeled tetrazine is administered to the subject within 24 hours, up to and including one day, up to and including two days, up to and including three days, up to and including four days, up to and including five days, up to and including six days, up to and including seven days, up to and including eight days, up to and including nine days, up to and including ten days, up to and including eleven days, up to and including twelve days, up to and including thirteen days, up to and including fourteen days, up to and including fifteen days, up to and including sixteen days, up to and including seventeen days, up to and including eighteen days, up to and including nineteen days, or up to and including twenty days after administration of the ASO linked to a trans-cyclooctene. In certain instances, the radiolabeled tetrazine is administered between about one and about two days, between about one and about three days, between about one and about four days, between about one and about five days, between about one and about six days, between about one and about seven days, between about one and about ten days, between about one and about fourteen days, between about one and about twenty days, between about one and about twenty four days, or between about one and about thirty two days after administration of the ASO linked to a trans-cyclooctene. In one instance, the radiolabeled tetrazine is administered to the subject about 24 hours after the administration of the ASO linked to a trans-cyclooctene. In some instances, the radiolabeled tetrazine is administered to the subject at or about 12, 13, 14, 15, 16, 17, 18, 19, 20, 21, 22, 23, 24, 25, 26, 27, 28, 29, 30, 31, 32, 33, 34, 35, or 36 hours after the administration of the ASO linked to a trans-cyclooctene.

In some instances, an additional step is added before the injection of the radioligand, specifically, the administration of a clearing agent designed to accelerate the removal of residual targeting agent (i.e., any targeting agent that is not bound to a target) from the bloodstream. See e.g., A Tetrazine-labeled dextran [Meyer, J.-P., et al. (2018). “Bioorthogonal Masking of Circulating Antibody-TCO Groups Using Tetrazine-Functionalized Dextran Polymers.” Bioconjugate Chemistry 29(2): 538-545]; and a galactose-albumin-tetrazine [Rossin et al., J. Nucl. Med., 54(11):1989-1995 (2013)].

In certain instances, after the administration of the radiolabeled tetrazine, the subject is imaged. In certain instances, after the administration of the radiolabeled tetrazine, the subject is imaged based on the PK of the radiolabeled tetrazine. In some cases, the imaging is done about 15 minutes, about 30 minutes, about 45 minutes, about 60 minutes, about 75 minutes, about 90 minutes, about 105 minutes, about 120 minutes, about 135 minutes, or about 150 minutes after injection of the radiotracer. In some cases, the imaging is done about half-an-hour to about 1 hour, about 1 hour to about one and a half hours, about two hours, about three hours, about four hours, or about five hours after injection of the radiotracer. In certain cases, imaging is conducted by any suitable diagnostic imaging method known in the art including but not limited to Positron Emission Tomography (PET), Positron emission tomography-computed tomography (PET-CT), Single Photon Emission Computed Tomography (SPECT), Single-photon emission computed tomography (SPECT-CT), Planar gamma camera, X-ray CT, planar X-ray, Magnetic Resonance Imaging (MRI), optical imager, or other diagnostic imaging technique.

In certain instances, the subject includes any human or non-human mammal. In certain non-limiting embodiments, the subject is a non-human primate, sheep, a dog, a cat, a rabbit, a horse, a cow, or a rodent.

In certain instances, the subject is a human subject. In certain cases, the human subject is a pediatric patient. In certain cases, the human subject is an infant. In certain instances, the human subject is an adult patient (i.e., 18 years or older). In some cases, the human subject has a CNS disorder. In certain cases, the CNS disorder is a synucleinopathy or a tauopathy. In some cases, the CNS disorder is spinal muscular atrophy (SMA), amyotrophic lateral sclerosis (ALS), Parkinson's disease, Alzheimer's disease, Huntington's disease, Angelman syndrome, frontotemporal dementia (FTD), Creutzfeldt-Jakob disease, spinocerebellar ataxia type 3 (SCA3), or Menkes disease.

In some instances, the distribution of the antisense oligonucleotide is evaluated in the CNS (e.g., cortex, striatum, thalamus, substantia nigra, cerebellum) of the human subject.

### Methods of Assessing ASO Concentration

Also featured are methods for determining the concentration of a biomolecule (e.g., an ASO) in a target region (e.g., the brain and/or spinal cord) of a subject (e.g., human). The method involves administering a biomolecule (e.g., ASO) linked to a trans-cyclooctene described herein to the subject. In some cases, the ASO linked to a trans-cyclooctene is administered intravenously. In certain cases, particularly where the distribution of the ASO is assessed in the brain and/or spinal cord, the ASO linked to a trans-cyclooctene is administered intrathecally. In certain cases, the ASO linked to a trans-cyclooctene is formulated in PBS or a-CSF. Following this administration, the subject is administered a radiolabeled tetrazine disclosed herein. In some instances, the radiolabeled tetrazine is a central nervous system penetrant compound. In some instances, the tetrazine is radiolabeled with a radionuclide that decays exclusively or almost exclusively through positron emission. In some instances, the tetrazine is radiolabeled with a radionuclide that has a short half-life (less than 12 hours) or a moderate half-life (about 12 to 18 hours). In some instances, the tetrazine is radiolabeled with a fluorine-18, carbon-11, or gallium-68. In some instances, the tetrazine compound does not include a chelator. In some cases, the radiolabeled tetrazine is administered intravenously. In certain cases, the radiolabeled tetrazine is formulated in PBS or a-CSF. In some instances, an additional step is added before the injection of the radioligand, specifically, the administration of a clearing agent designed to accelerate the removal of residual targeting agent from the bloodstream. The method further involves imaging the distribution of the biomolecule in the subject and deriving a tissue concentration of the biomolecule (e.g., ASO) in the subject (e.g., brain and/or spinal cord of the subject).

The timing of when the radiolabeled tetrazine is administered depends on the half-life of each of the ASO and the trans-cyclooctene. In some cases, the radiolabeled tetrazine is administered to the subject within 24 hours, up to and including one day, up to and including two days, up to and including three days, up to and including four days, up to and including five days, up to and including six days, up to and including seven days, up to and including eight days, up to and including nine days, up to and including ten days, up to and including eleven days, up to and including twelve days, up to and including thirteen days, up to and including fourteen days, up to and including fifteen days, up to and including sixteen days, up to and including seventeen days, up to and including eighteen days, up to and including nineteen days, or up to and including twenty days after administration of the ASO linked to a trans-cyclooctene. In certain embodiments, the radiolabeled tetrazine is administered between about one and about two days, between about one and about three days, between about one and about four days, between about one and about five days, between about one and about six days, between about one and about seven days, between about one and about ten days, between about one and about fourteen days, between about one and about twenty days, between about one and about twenty four days, or between about one and about thirty two days after administration of the ASO linked to a trans-cyclooctene. In one instance, the radiolabeled tetrazine is administered to the subject about 24 hours after the administration of the ASO linked to a trans-cyclooctene. In some instances, the radiolabeled tetrazine is administered to the subject at or about 12, 13, 14, 15, 16, 17, 18, 19, 20, 21, 22, 23, 24, 25, 26, 27, 28, 29, 30, 31, 32, 33, 34, 35, or 36 hours after the administration of the ASO linked to a trans-cyclooctene.

In some cases, the imaging is done about 15 minutes, about 30 minutes, about 45 minutes, about 60 minutes, about 75 minutes, about 90 minutes, about 105 minutes, about 120 minutes, about 135 minutes, or about 150 minutes after injection of the radiotracer. In some cases, the imaging is done about half-an-hour to about 1 hour, about 1 hour to about one and a half hours, about two hours, about three hours, about four hours, or about five hours after injection of the radiotracer. In certain cases, imaging is conducted by any suitable diagnostic imaging method known in the art including but not limited to Positron Emission Tomography (PET), Positron emission tomography-computed tomography (PET-CT), Single Photon Emission Computed Tomography (SPECT), Single-photon emission computed tomography (SPECT-CT), Planar gamma camera, X-ray CT, planar X-ray, Magnetic Resonance Imaging (MRI), optical imager, or other diagnostic imaging technique.

From the imaging data, an uptake value of radiolabeled tetrazine for each region-of-interest can be calculated. This value can then be applied to either an equation or a reference lookup table that has been assembled empirically to provide a corresponding concentration of the biomolecule (e.g., ASO) in the tissue.

In some instances, the concentration of the biomolecule (e.g., ASO) is evaluated in the CNS (e.g., cortex, striatum, thalamus, substantia nigra, cerebellum) of the subject.

In certain instances, the subject is a human subject. In certain cases, the human subject is a pediatric patient. In certain cases, the human subject is an infant. In certain cases, the human subject is an adult patient (i.e., 18 years or older). In some cases, the human subject has a CNS disorder. In certain cases, the CNS disorder is a synucleinopathy or a tauopathy. In some cases, the CNS disorder is spinal muscular atrophy (SMA), amyotrophic lateral sclerosis (ALS), Parkinson's disease, Alzheimer's disease, Huntington's disease, Angelman syndrome, frontotemporal dementia (FTD), Creutzfeldt-Jakob disease, spinocerebellar ataxia type 3 (SCA3), or Menkes disease.

### Compositions

The present disclosure also provides pharmaceutical compositions comprising an ASO-TCO and/or radiolabeled tetrazine compounds described herein. In certain instances, such pharmaceutical compositions comprise or consist of a sterile saline solution and ASO-TCO and/or radiolabeled tetrazine compounds. In some cases, such pharmaceutical compositions are sterile, buffered, isotonic solutions. In some cases, the pharmaceutical compositions are preservative-free.

The ASO-TCO and/or radiolabeled tetrazine compounds described herein may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Antisense oligonucleotide compounds or a salt thereof can be utilized in pharmaceutical compositions by combining such compounds with a suitable pharmaceutically acceptable diluent or carrier. In certain instances, the pharmaceutically acceptable diluent is phosphate-buffered saline (PBS). In certain embodiments, the pharmaceutically acceptable diluent is artificial cerebrospinal fluid (aCSF).

In certain embodiments, the aCSF formulation has a pH of 7.2. The pH of the composition can be adjusted, if necessary, with hydrochloric acid or sodium hydroxide during compounding.

Pharmaceutical compositions comprising antisense compounds encompass any pharmaceutically acceptable salts, esters, or salts of such esters, and hydrates thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

In some instances, the ASO-TCO and/or radiolabeled tetrazine compounds are formulated for intravenous administration. In some instances, the ASO-TCO and/or radiolabeled tetrazine compounds are formulated for intrathecal administration. In certain cases, the ASO-TCO and radiolabeled tetrazine compounds are formulated in phosphate buffered saline (PBS). In other cases, the ASO-TCO and radiolabeled tetrazine compounds are formulated in artificial cerebrospinal fluid (a-CSF). In yet other cases, the ASO-TCO and radiolabeled tetrazine compounds are formulated in sterile water for injection.

### Kits

The disclosure further provides kits that can be used to practice the methods disclosed herein. For example, a kit can comprise at least one targeting probe (e.g., an ASO-TCO described herein) and/or at least one labeling probe (e.g., a radiolabeled tetrazine described herein). In certain embodiments, a kit can optionally comprise instructions on how to use the kit for molecular imaging. In certain instances, a kit can further comprise an administration device such as a syringe and/or catheter and/or introducer sheath.

The disclosure further provides kits for preparing the targeting probe and/or labeling probe. In certain instances, the kit of the present invention contains the targeting probe (in dry or liquid form) and/or the labeling probe (in dry or liquid form) for application on the biomaterial. When the probe is provided in dry form, the kit can contain the appropriate buffer or solvent to prepare a solution or composition.

### Definitions

The term “about” in the context of an amount, e.g., about X mg means+/−10%, so “about 50 mg” encompasses 45 mg to 55 mg. The term “about” in the context of X days means+/−3 days, so “about 10 days” encompasses 7 to 13 days. The term “about” in the context of X months means+/−1 week, so “about 4 months” encompasses a week before and after the 4 month mark. The term “about” in the context of X hours means+/−3 hours, so “about 10 hours” encompasses 7 to 13 hours. The term “about” in the context of X minutes means+/−10 minutes, so “about 100 minutes” encompasses 90 to 110 minutes. The term “about” in the context of X temperature means+/−3° C.

The term “alkyl” refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. The alkyl group can containing from 1 to 6 carbon atoms, from 1 to 4 carbon atoms, from 1 to 3 carbon atoms, or 1 to 2 carbon atoms. Examples of alkyl moieties include chemical groups such as methyl, ethyl, n-propyl, isopropyl, n-butyl, tert-butyl, isobutyl, sec-butyl, and the like.

The term “alkylene” refers to a divalent alkyl linking group. An alkylene group formally corresponds to an alkane with two C—H bond replaced by points of attachment of the alkylene group to the remainder of the compound. Examples of alkylene groups include ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “haloalkyl” refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CH₂F, CCl₃, CHCl₂, C₂Cl₅ and the like. In some embodiments, the haloalkyl group is a fluoroalkyl group such as CF₃, CHF₂, or CH₂F.

The term “heteroaryl” refers to a monocyclic heterocycle having at least one heteroatom ring member selected from sulfur, oxygen and nitrogen. The heteroaryl ring can have 1, 2, 3 or 4 heteroatom ring members independently selected from nitrogen, sulfur and oxygen. Any ring-forming N in a heteroaryl moiety can be an N-oxide. The heteroaryl can have 5-6 ring atoms and 1 or 2 heteroatom ring members independently selected from nitrogen, sulfur and oxygen. In some instances, the heteroaryl is a five-membered or six-membered heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, isoxazolyl, thiazolyl, imidazolyl, furanyl, thiophenyl, and the like.

The present disclosure also includes salts of the compounds described herein including pharmaceutically acceptable salts. The term “pharmaceutically acceptable salts” refers to derivatives of the disclosed compounds wherein the parent compound is modified by converting an existing acid or base moiety to its salt form. Examples of pharmaceutically acceptable salts include mineral or organic acid salts of basic residues such as amines; alkali or organic salts of acidic residues such as carboxylic acids; and the like. The pharmaceutically acceptable salts can include the non-toxic salts of the parent compound formed, e.g., from non-toxic inorganic or organic acids. The pharmaceutically acceptable salts can be synthesized from the parent compound which contains a basic or acidic moiety by conventional chemical methods. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002).

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### EXAMPLES

### Example 1: Synthesis of Compound 1

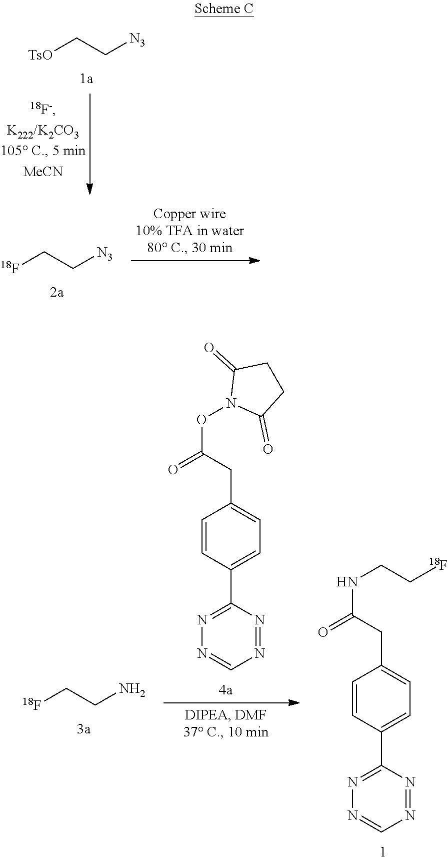

Compound 1 was prepared according to the procedures in Scheme B. Materials and reagents used in the preparation of Compound 1 are as follows:

| Compound | Manufacturer | Product # |
|---|---|---|
| [¹⁸F]Fluoride in target water | PETNET | n/a |
| BI-Tz-cpdB reference standard | n/a | n/a; MW = 261.26 |
| 2-azidoethyl 4-methylbenzenesulfonate | Enamine | EN300-63896 |
| Tz NHS ester | Click Chem | 1127-100 |
| Acetonitrile (MeCN)-HPLC grade | Fisher | A998-4 |
| Ammonium acetate | Amresco | 0103-50 G |
| N,N-Dimethylformamide | Sigma | 227056-100 ML |
| (DMF)-Anhydrous | | |
| N,N-Diisopropylethylamine, purified by | Sigma | 387649-100 ML |
| redistillation (DIPEA) | | |
| Copper wire, 0.127 mm dia., 99.999% | Alfa Aesa | 00100G7 |
| (metals basis) | | |
| Ethanol (200 proof) | Fisher | BP2818-500 |
| Kryptofix 2.2.2 | Sigma | 291110-1 G |
| Potassium Carbonate | Sigma | 367877-10 G |
| Hydrochloric Acid Solution, 1N | Fluka | 717631 L |
| Trifluoroacetic Acid, HPLC grade | EMD | TX12766 |
| Dulbecco's phosphate buffered saline | gibco | 14190-144 |
| Water-HPLC grade | Fisher | W5-4 |
| Waters QMA Sep-Pak (46 mg) | Waters | 186004540 |
| Phenomenex Strata-X, Reversed Phase, | Phenomenex | 8B-S100-TAL |
| 30 mg/1 mL | | |

The HPLC methods are as follows.

| | Semi-Preparative | Analytical |
|---|---|---|
| HPLC Column | Phenomenex Gemini NX | Water Xbridge C18, |
| | C18 110 Å, 5 μ, LC | 5 μm, LC Column |
| | Column 250 × 10 mm | 150 × 4.6 mm |
| Eluent | 10 mM NH₄OAc | 10 mM NH₄OAc |
| | (10 mM, pH 7)/ | (10 mM, pH 7)/ |
| | MeCN (85/15) | MeCN (80/20) |
| Flow rate | 5 | 1 |
| (mL/min) | | |
| UV setting (nm) | 280 | 270 |
| Expected Rt | 26.5 | [¹⁸F]FEAzide = 6.0; |
| (min) | | [¹⁸F]FEAmine = 2.0; |
| | | [¹⁸F]BI-Tz-cpdB = 7.9 |

The preparation of Compound 1 was carried out using a radiosynthesizer, TRACERlab FX2N. Pre-synthesis set up includes:
1. Install argon gas line onto Vial 2-top.
2. Make sure Ar gas flow is @ ˜20 mL/min (test from VX2 to Al₂O₃ 1 top by a flow meter).
3. Connect a 2″ 21G needle to Al₂O₃ 1 top and insert the needle to the bottom of a vented (1″ 21G needle) 0.3 mL V-vial containing MeCN (0.15 mL).
4. Place the 0.3 mL V-vial in an ice bath.

### GE Tracerlab FX2N Vial Set-up

| | |
|---|---|
| Vial 1 | Kryptofix 2.2.2 (7.5 mg) in MeCN (0.4 mL) + |
| | potassium carbonate (0.75 mg) in water (0.4 mL) |
| Vial 2 | MeCN (0.15 mL) |
| Vial 3 | Azide precursor (3 μL) in anhydrous MeCN (0.5 mL) |
| SPE 1 | Waters QMA Sep-Pak (46 mg) |

### Preparation of Compound 2a

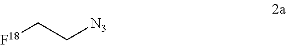

[¹⁸F]Fluoride (1215 mCi at start of synthesis) produced via the ¹⁸O(p,n)¹⁸F nuclear reaction by a cyclotron equipped with a high-yield oxygen-18 water target, was purchased from PETNET. The [¹⁸F]fluoride in ˜1 mL of [¹⁸O]H₂O was trapped on a Waters QMA cartridge pre-conditioned with HPLC-grade water (5 mL), to remove [¹⁸O]H₂O. [¹⁸F]Fluoride was eluted into Reactor 1 by passing K₂₂₂/K₂CO₃ solution (7.5 mg/0.75 mg in 0.4 mL/0.4 mL of HPLC-grade acetonitrile/water) through the cartridge. The [¹⁸F]fluoride was then dried by heat (70° C.) and a stream of nitrogen under full vacuum for 5 min followed by only full vacuum at 100° C. for 5 min. After drying, the solution of azide precursor (Compound 1a, 3 μL) in anhydrous MeCN (0.5 mL) was added and the resulting solution was heated at 105° C. with stirring for 5 min. The reaction mixture was then cooled to 30° C. followed by adding MeCN (0.15 mL). The 2-[¹⁸F]fluoroethyl azide ([¹⁸F]FEAzide, Compound 2a) was then distilled into a 0.3 mL V-vial containing ice-cold MeCN (0.15 mL) with heat (130° C.) and a stream of Argon (20 mL/min). The distilled [¹⁸F]FEAzide (Compound 2a, 360 mCi @ 10:57) in MeCN (328 μL) was analyzed by HPLC and the radiochemical purity (RCP) is 99%.

### Preparation of Compound 3a

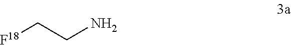

The distilled [¹⁸F]FEAzide (Compound 2a, 150 μL; 164.7 mCi @ 10:57) from the previous step was added to a 0.3 mL V-vial containing a copper wire plug (˜100 mg) and 10% TFA in water (150 μL). The resulting mixture was reacted at 80° C. for 30 min to afford [¹⁸F]FEAmine (Compound 3a). HPLC shows 86% conversion from [18F]FEAzide (Compound 2a). The [¹⁸F]FEAmine (Compound 3a) in 50:50=MeCN:10% TFA in water was used in the subsequent coupling reaction without further purification.

### Preparation of Compound 1

Three coupling conditions were tested, and all were reacted at 37° C. for 10 min.
(a) 150 μL of [¹⁸F]FEAmine (Compound 3a)+4 mg Tz NHS ester (Compound 4a) in DMF (0.3 mL)+DIPEA (80 μL)
(b) 300 μL of [¹⁸F]FEAmine (Compound 3a)+4 mg Tz NHS ester (Compound 4a) in DMF (0.3 mL)+DIPEA (100 μL)
(c) Dry [¹⁸F]FEAmine ((Compound 3a) evaporate TFA, water and MeCN under vacuum and a nitrogen stream)+4 mg Tz NHS ester (Compound 4a) in DMF (0.3 mL)+DIPEA (30 μL)

The reaction mixture was analyzed using analytical HPLC and the results suggest condition (a) provided best coupling yield. Condition (a) provided 47.5% yield; condition (b) provided 27.7% yield; and condition (c) provided 29.6% yield.

The reaction mixture from condition (a) was worked up by adding 4.5 mL of 10 mM NH₄OAc (final mixture pH ˜11) or 4 mL of 10 mM NH₄OAc+0.35 mL 1N HCl (final mixture pH ˜4). The reaction mixture after work-up was analyzed by analytical HPLC and the results suggest the reaction mixture should be worked up with 4 mL of 10 mM NH₄OAc+0.35 mL 1N HCl. Specifically, Compound 1 was not observed in the HPLC chromatogram when the reaction mixture was worked up by adding 4.5 mL of 10 mM NH₄OAc (final mixture pH ˜11). There was 48% of Compound 1 in the HPLC chromatogram when the reaction mixture was worked by adding 4 mL of 10 mM NH₄OAc+0.35 mL 1N HCl (final mixture pH ˜4).

Compound 1 synthesis on FX-FN

### GE Tracerlab FX-FN Vial Set-up

| | |
|---|---|
| Vial 1 to 5 | empty |
| Vial 6 | 10 mM NH₄OAc (4 mL) + 1N HCl (0.35 mL) |
| Vial 7 | empty |
| Vial 8 | EtOH (0.5 mL) |
| Vial 9 | water (5 mL) |
| HPLC dilution flask | water (40 mL) |
| Reactor | 0.15 mL of [¹⁸F]FEAmine + 4 mg Tz NHS |
| | ester in DMF (0.3 mL) + DIPEA (80 μL) |
| SPE3 | Phenomenex Strata-X, Reversed Phase, |
| | 30 mg/1 mL |

The reactor on FX-FN was loaded with [¹⁸F]FEAmine (Compound 3a, 0.15 mL; 67 mCi @ 11:32), 4 mg of Tz NHS ester (Compound 4a) in DMF (0.3 mL) and DIPEA (80 μL) and the resulting mixture was reacted at 37° C. for 10 min followed by diluting with 4 mL of 10 mM NH₄OAc and 0.35 mL of 1 N HCl. The diluted mixture was transferred to loop-loading vial followed by loading onto the semi-preparative HPLC for purification as described above.

The semi-preparative trace showed the product peak (Rt˜26 min), which was collected into the HPLC dilution flask and diluted with HPLC-grade water (40 mL). The purified Compound 1 was then trapped on a Strata-X, Reversed Phase, 30 mg/l mL cartridge (PN 8B-S100-TAL), pre-conditioned with ethanol (5 mL) and water (5 mL) followed by washing with water (5 mL). The trapped Compound 1 was eluted with ethanol (0.5 mL) into 8 mL vail and the ethanol was evaporated at 37° C. with a stream of argon. Compound 1 was reconstituted with 1 mL of 1×DPBS and submitted for quality control testing.

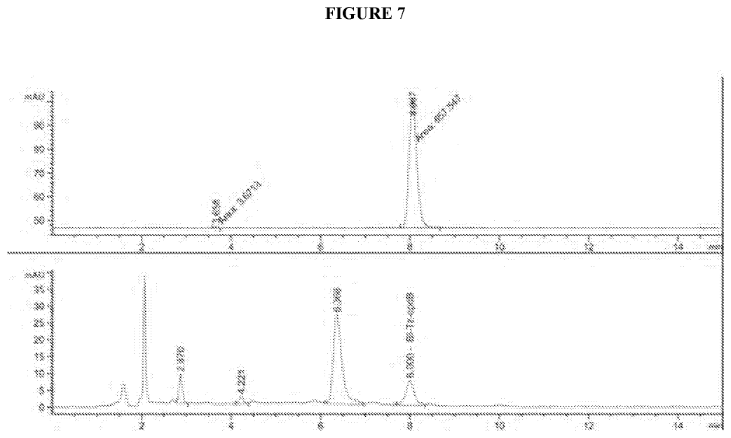

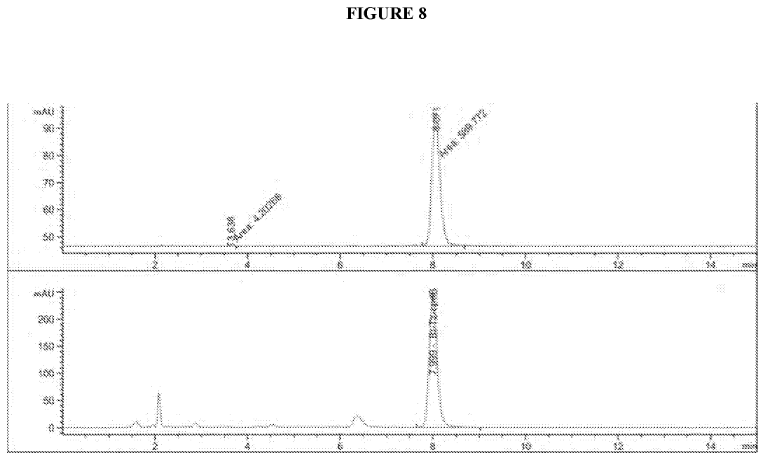

Chemical and radiochemical purities/identities are analyzed using an Agilent 1100 HPLC equipped with a radioactivity detector and an ultraviolet (UV) detector (See FIG. 7 and FIG. 8). Radiochemical purity for the dose was >99%, and identity is confirmed by comparing the retention time of the radiolabeled product with that of the corresponding unlabeled reference standard. RCY from [18F]fluoride 1.8%/

Compound 1 has the following in silico and in vitro features:

| In Silico | |
|---|---|
| MW | 261 |
| cLogP | −0.95 |
| TPSA | 81 |
| cnsMPO | 5.83 |
| cnsPET MPO | 5.24 |
| In Vitro | |
| MDCK-BCRP | 0.51 |
| (B-A/A-B) | |
| PPB (rat) | 37.5% |
| | unbound |
| BPB (rat) | 51% |
| | unbound |
| Microsome | 84 min |
| Stability t_{1/2} | |

### Example 2: Synthesis of 2-(4-(1,2,4,5-tetrazin-3-yl)phenyl)-N-(2-fluoroethyl)acetamide (Compound 1′)

### Preparation of 2-(4-(1,2,4,5-tetrazin-3-yl)phenyl)acetic acid

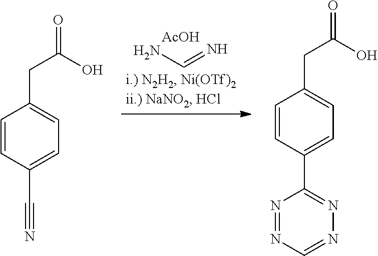

A 40 mL sealed tube was charged with a solution of 2-(4-cyanophenyl)acetic acid (2.00 g, 12.4 mmol, 1.00 eq), methanimidamide acetate (6.46 g, 62.1 mmol), Ni(OTf)₂ (221 mg, 620 umol) in N₂H₄.H₂O (19.0 g, 372 mmol, 18.5 mL). The mixture was stirred at 35° C. for 12 h, a solution of NaNO₂ (17.1 g, 248 mmol, 20.0 eq) in H₂O (10 mL) was added into the mixture at 5° C. and followed by slow addition of 1 M HCl during which the solution turned bright red in color and gas evolved, addition of 1 M HCl continued until gas evolution ceased and the pH value is 3. The mixture was extracted with EtOAc (100 mL×4), and the organic layer was washed with brine (100 mL×2), dried over Na₂SO₄, filtered and concentrated. The crude product was used in the next step without purification. The title compound (2.00 g) was obtained as red solid. LCMS: m/z=217.2 [M+H]⁺. ¹HNMR: (400 MHz, CDCl₃) δ 10.23 (s, 1H), 8.64-8.60 (m, 2H), 7.55 (d, J=8.4 Hz, 2H), 3.80 (s, 2H).

### Preparation of 2-(4-(1,2,4,5-tetrazin-3-yl)phenyl)-N-(2-fluoroethyl)acetamide (Compound 1′)

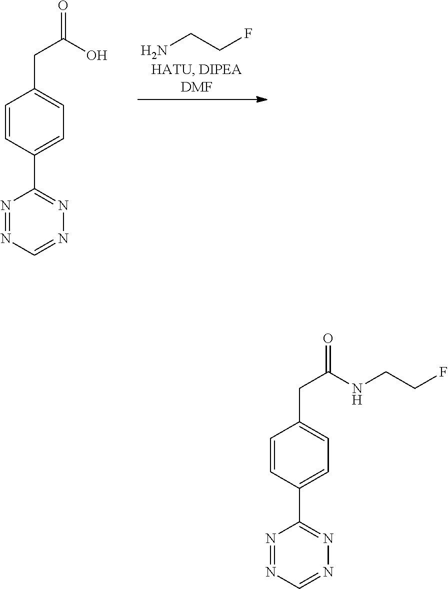

A 100 mL single necked round bottom flask was charged with a solution of 2-(4-(1,2,4,5-tetrazin-3-yl)phenyl)acetic acid (2.00 g, 9.25 mmol), 2-fluoroethanamine (1.01 g, 10.2 mmol, HCl), HATU (5.28 g, 13.9 mmol), and DIPEA (3.59 g, 27.8 mmol, 4.83 mL) in DMF (20 mL), the mixture was stirred at 25° C. for 2 h. The mixture was added into the ice-water (100 mL), the mixture was extracted with EtOAc (30 mL×3), the organic layer was washed with brine (30 mL×3), dried over Na₂SO₄, filtered and concentrated. The residue was purified by prep HPLC column: Phenomenex luna C18 150*40 mm*15 um; mobile phase: [water (0.225% FA)-ACN]; B %: 5%-35%, 9 min to give the desired compound. The title compound was obtained as red solid (230 mg, 9% yield). LCMS: m/z=262.2 [M+H]⁺. ¹HNMR: (400 MHz, CDCl₃) δ 10.24 (s, 1H), 8.64 (d, J=8.4 Hz, 2H), 7.55 (d, J=8.4 Hz, 2H), 5.87 (br s, 1H), 4.55 (t, J=4.8 Hz, 1H), 4.44 (t, J=4.8 Hz, 1H), 3.72 (s, 2H), 3.63 (q, J=4.8 Hz, 1H), 3.56 (q, J=4.8 Hz, 1H).

### Example 3: Synthesis of 3-(4-(((1-(2-fluoroethyl)-1H-1,2,3-triazol-4-yl)methoxy)methyl)phenyl)-1,2,4,5-tetrazine (Compound 2′)

### Preparation of 4-(((4-bromobenzyl)oxy)methyl)-1H-1,2,3-triazole

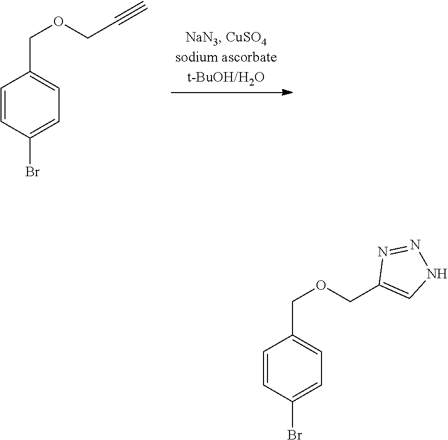

To a solution of 1-bromo-4-((prop-2-yn-1-yloxy)methyl)benzene (60.0 g, 266 mmol) in t-BuOH (300 mL) and H₂O (300 mL) was added sodium ascorbate (58.7 g, 296 mmol) and CuSO₄ (14.1 g, 88.8 mmol). Then, NaN₃ (19.3 g, 296 mmol) was added and the mixture was stirred at 70° C. for 1 h. Dichloromethane (1 L) was added to the mixture, the suspension was filtered and the filtrate was extracted with dichloromethane (500 mL×3), the organic layer was dried over Na₂SO₄, filtered and concentrated. The residue was purified by silica gel chromatography eluted with dichloromethane:methanol (100:1˜50:1, 5 L) to give the title compound (16.0 g, 20% yield) as light yellow solid. ¹HNMR: (400 MHz, CDCl₃) δ 12.62 (br s, 1H), 7.80-7.63 (m, 1H), 7.53-7.42 (m, 2H), 7.24 (d, J=8.4 Hz, 2H), 4.71 (s, 2H), 4.55 (s, 2H).

### Preparation of tert-butyl 4-(((4-bromobenzyl)oxy)methyl)-2H-1,2,3-triazole-2-carboxylate

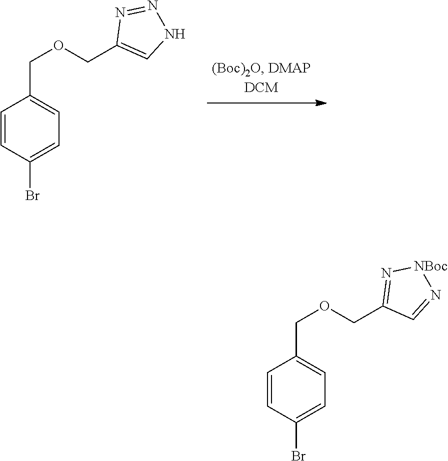 [SMILES: Brc1ccc(COCc2c[nH]nn2)cc1 ; CC(C)(C)OC(=O)n1ncc(COCc2ccc(Br)cc2)n1]

A 40 mL sealed tube was charged with a solution of 4-(((4-bromobenzyl)oxy)methyl)-1H-1,2,3-triazole (10.0 g, 37.3 mmol) and (Boc)₂O (9.77 g, 44.7 mmol) in DCM (100 mL). DMAP (455 mg, 3.73 mmol) was added to the mixture, which was subsequently stirred at 20° C. for 1 h. The mixture was washed with citric acid (50 mL×4), brine (50 mL×3), the organic layer was dried over Na₂SO₄, filtered and concentrated. The crude product was used in the next step without purification. The title compound (14.0 g) was obtained as yellow oil. ¹HNMR: (400 MHz, CDCl₃) δ 7.87 (s, 1H), 7.52-7.45 (m, 2H), 7.25-7.18 (m, 2H), 4.71 (s, 2H), 4.58-4.51 (m, 2H), 1.72-1.66 (m, 9H).

### Preparation of 4-(((1H-1,2,3-triazol-4-yl)methoxy)methyl)benzonitrile

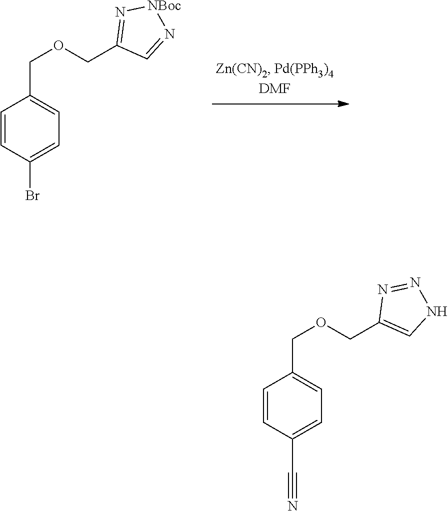 [SMILES: CC(C)(C)OC(=O)n1ncc(COCc2ccc(Br)cc2)n1 ; N#Cc1ccc(COCc2c[nH]nn2)cc1]

To a solution of tert-butyl 4-(((4-bromobenzyl)oxy)methyl)-2H-1,2,3-triazole-2-carboxylate (13.0 g, 35.3 mmol) in DMF (130 mL) under a nitrogen atmosphere was added Pd(PPh₃)₄ (8.16 g, 7.06 mmol) and Zn(CN)₂ (8.29 g, 70.6 mmol). The reaction mixture was stirred at 100° C. for 12 h. The mixture was diluted with ethyl acetate (100 mL) and H₂O (500 mL). The mixture was filtered over celite and the organic layer was separated, washed with brine (100 mL), dried over Na₂SO₄, filtered and concentrated. The residue was purified by prep-HPLC (EW18117-27-P1C) column: Phenomenex Synergi Max-RP 250×50 mm×10 um; mobile phase: [water(0.225% FA)-ACN]; B %: 15%-40%, 20 min to give the desired compound. The title compound (1.7 g, 22% yield) was obtained as yellow solid. LCMS: m/z=215.2 [M+H]⁺. ¹HNMR: (400 MHz, CDCl₃) δ 7.77 (s, 1H), 7.68-7.62 (m, 2H), 7.46 (d, J=8.4 Hz, 2H), 4.76 (s, 2H), 4.69-4.61 (m, 2H).

### Preparation of 3-(4-(((1H-1,2,3-triazol-4-yl)methoxy)methyl)phenyl)-1,2,4,5-tetrazine

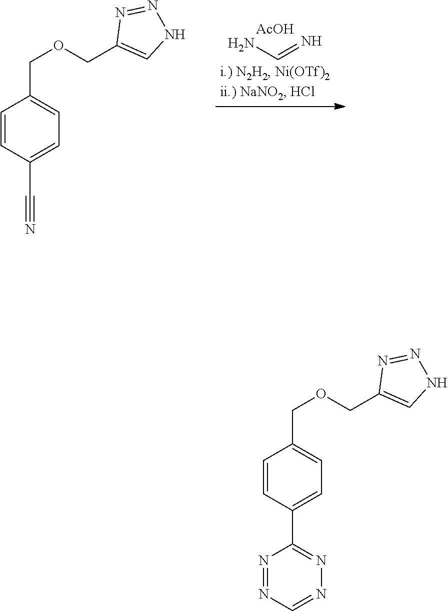

A 100 mL single necked round bottom flask was charged with a solution of 4-(((1H-1,2,3-triazol-4-yl)methoxy)methyl)benzonitrile (1.70 g, 7.94 mmol), methanimidamide acetate (8.26 g, 79.3 mmol), Ni(OTf)₂ (1.42 g, 3.97 mmol) in N₂H₄.H₂O (20.3 g, 396 mmol, 19.7 mL). The mixture was stirred at 35° C. for 12 h, a solution of NaNO₂ (10.9 g, 158 mmol) in H₂O (17 mL) was added into the mixture at 5° C. and followed by slow addition of 1 M HCl during which the solution turned bright red in color and gas evolved, addition of 1 M HCl continued until gas evolution ceased and the pH value is 3. The mixture was extracted with EtOAc (100 mL×3), the organic layer was washed with brine (100 mL×2), dried over Na₂SO₄, filtered and concentrated. The residue was purified by prep. HPLC column: Phenomenex Synergi C18 150*25*10 um; mobile phase: [water(0.225% FA)-ACN]; B %: 38%-68%, 10 min to give the crude product, the crude product was purified by prep. HPLC column: Phenomenex luna C18 150*25 mm*10 um; mobile phase: [water(0.1% TFA)-ACN]; B %: 16%-46%, 10 min. The residue was triturated with MeOH (5 mL) to give the desired compound. The title compound (105 mg, 5% yield) was obtained as red solid. LCMS: m/z=270.2[M+H]⁺. ¹HNMR: (400 MHz, DMSO-d6) δ 15.45-14.73 (m, 1H), 10.73-10.48 (m, 1H), 8.50 (d, J=8.4 Hz, 2H), 8.25-7.80 (m, 1H), 7.64 (d, J=8.4 Hz, 2H), 4.69 (s, 4H). 3-(4-(((1H-1,2,3-triazol-4-yl)methoxy)methyl)phenyl)-1,2,4,5-tetrazine can be used to prepare Compound 2′ using methods that are suitable for installing ethylfluoro group to the triazole ring.

### Preparation of 4-(((4-bromobenzyl)oxy)methyl)-1-(2-fluoroethyl)-1H-1,2,3-triazole

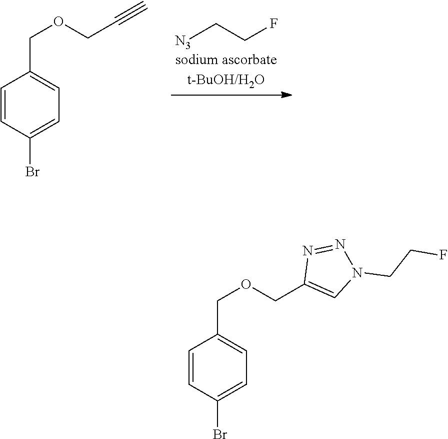 [SMILES: C#CCOCc1ccc(Br)cc1 ; [N-]=[N+]=NCCF ; FCCn1cc(COCc2ccc(Br)cc2)nn1]

A 250 mL single necked round bottom flask was charged with a solution of 1-bromo-4-((prop-2-yn-1-yloxy)methyl)benzene (5.00 g, 22.2 mmol), 1-azido-2-fluoroethane (2.18 g, 24.4 mmol), CuSO₄ (1.42 g, 8.89 mmol) and sodium ascorbate (4.84 g, 24.4 mmol) in t-BuOH (30 mL) and H₂O (30 mL). The mixture was stirred at 70° C. for 1 h. The mixture was cooled to r.t, EtOAc (100 mL) and water (100 mL) were added into the mixture, the suspension was filtered and the filtrate was extracted EtOAc (100 mL×2), the organic layer was washed with brine (100 mL×2), dried over Na₂SO₄, filtered, and concentrated. The residue was purified by silica gel chromatography eluted with Petroleum ether:Ethyl acetate (10:1˜1:2, 3 L) to give the desired compound. The title compound (5.00 g, 71% yield) was obtained as yellow oil. ¹HNMR: (400 MHz, CDCl₃) δ 7.67 (s, 1H), 7.46 (d, J=8.4 Hz, 2H), 7.23 (d, J=8.4 Hz, 2H), 4.85 (t, J=4.4 Hz, 1H), 4.74-4.67 (m, 4H), 4.66-4.61 (m, 1H), 4.55 (s, 2H).

### Preparation of 4-(((1-(2-fluoroethyl)-1H-1,2,3-triazol-4-yl)methoxy)methyl)benzonitrile

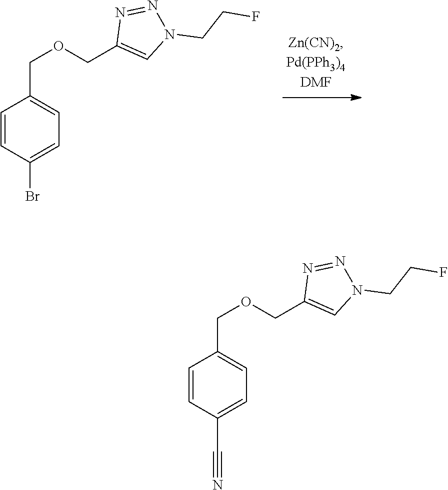 [SMILES: FCCn1cc(COCc2ccc(Br)cc2)nn1 ; N#Cc1ccc(COCc2cn(CCF)nn2)cc1]

To a solution of 4-(((4-bromobenzyl)oxy)methyl)-1-(2-fluoroethyl)-1H-1,2,3-triazole (5.00 g, 15.9 mmol) in DMF (50 mL) under a nitrogen atmosphere was added Pd(PPh₃)₄ (3.68 g, 3.18 mmol, 0.20 eq) and Zn(CN)₂ (3.74 g, 31.8 mmol, 2.02 mL, 2.00 eq), the reaction mixture was stirred at 100° C. for 12 h. The mixture was diluted with ethyl acetate (50 mL) and H₂O (50 mL). The mixture was filtered over celite. The organic layer was separated, washed with brine (50 mL), dried over Na₂SO₄, filtered and concentrated. The residue was purified by prep-HPLC (EW18117-24-P1A) column: Phenomenex luna C18 250*50 mm*10 um; mobile phase: [water(0.225% FA)-ACN]; B %: 20%-50%, 15 min to give the desired compound. The title compound (2.00 g, 48% yield) was obtained as yellow solid. LCMS: m/z=261.2 [M+H]⁺. ¹HNMR: (400 MHz, CDCl₃) δ 7.71 (s, 1H), 7.63 (d, J=8.0 Hz, 2H), 7.46 (d, J=8.0 Hz, 2H), 4.91-4.84 (m, 1H), 4.77-4.71 (m, 4H), 4.69-4.63 (m, 3H).

### Preparation of 3-(4-(((1-(2-fluoroethyl)-1H-1,2,3-triazol-4-yl)methoxy)methyl)phenyl)-1,2,4,5-tetrazine (Compound 2′)

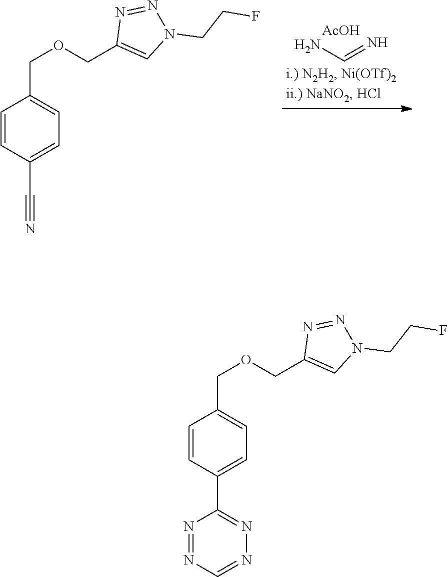

A 100 mL single necked round bottom flask was charged with a solution of 4-(((1-(2-fluoroethyl)-1H-1,2,3-triazol-4-yl)methoxy)methyl)benzonitrile (2.00 g, 7.68 mmol), methanimidamide acetate (8.00 g, 76.8 mmol), Ni(OTf)₂ (1.37 g, 3.84 mmol) in N₂H₄.H₂O (19.6 g, 384 mmol, 19.06 mL). The mixture was stirred at 35° C. for 12 h, a solution of NaNO₂ (10.6 g, 154 mmol) in H₂O (20 mL) was added into the mixture at 5° C. and followed by slow addition of 1 M HCl during which the solution turned bright red in color and gas evolved, addition of 1 M HCl continued until gas evolution ceased and the pH value is 3. The mixture was extracted with EtOAc (100 mL×3), the organic layer was washed with brine (100 mL×2), dried over Na₂SO₄, filtered and concentrated. The residue was purified by prep. HPLC column: column: Phenomenex luna C18 150*40 mm*15 um; mobile phase: [water(0.225% FA)-ACN]; B %: 15%-45%, 9 min to give the crude compound, the crude product was purified by prep. HPLC column: Phenomenex luna C18 150*25 mm*10 um; mobile phase: [water(0.1% TFA)-ACN]; B %: 22%-52%, 10 min to give the desired compound. The title compound (52.0 mg, 2% yield) was obtained as red solid. LCMS: m/z=331.2 [M+H]⁺. ¹HNMR: (400 MHz, DMSO-d6) δ 10.68-10.40 (m, 1H), 8.54-8.46 (m, 2H), 8.23 (s, 1H), 7.64 (d, J=8.4 Hz, 2H), 4.93-4.87 (m, 1H), 4.80-4.75 (m, 2H), 4.73-4.69 (m, 3H), 4.68 (s, 2H).

### Example 4: Synthesis of 3-(4-(((2-(2-fluoroethyl)-2H-1,2,3-triazol-4-yl)methoxy)methyl)phenyl)-1,2,4,5-tetrazine (Compound 3′)

### Preparation of 4-(((4-bromobenzyl)oxy)methyl)-2-(2-fluoroethyl)-2H-1,2,3-triazole

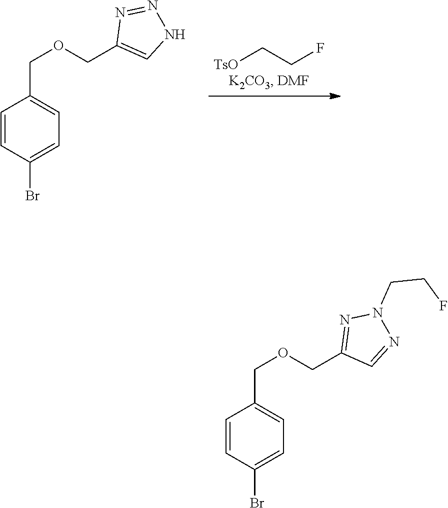 [SMILES: Brc1ccc(COCc2c[nH]nn2)cc1 ; Cc1ccc(S(=O)(=O)OCCF)cc1 ; FCCn1ncc(COCc2ccc(Br)cc2)n1]

A 8 mL sealed tube was charged with a solution of 4-(((4-bromobenzyl)oxy)methyl)-1H-1,2,3-triazole (1.00 g, 3.73 mmol) and K₂CO₃ (773 mg, 5.59 mmol) in DMF (10 mL), 2-fluoroethyl 4-methylbenzenesulfonate (895 mg, 4.10 mmol) was added into the mixture which was stirred at 20° C. for 12 h. Ice-water (20 mL) was added and the mixture was extracted with EtOAc (20 mL×3). The combined organic layers were washed with brine (20 mL×3), dried over Na₂SO₄, filtered and concentrated. The residue was purified by silica gel chromatography eluted with Ethyl acetate:Petroleum ether (10: 1-3:1, 1.5 L) to give the desired compound. The title compound (700 mg, 58% yield) was obtained as yellow oil. ¹HNMR: (400 MHz, CDCl₃) δ 7.66 (s, 1H), 7.53-7.44 (m, 2H), 7.28-7.21 (m, 2H), 5.00-4.82 (m, 2H), 4.79-4.67 (m, 2H), 4.64 (s, 2H), 4.55 (s, 2H).

### Preparation of 4-(((2-(2-fluoroethyl)-2H-1,2,3-triazol-4-yl)methoxy)methyl)benzonitrile

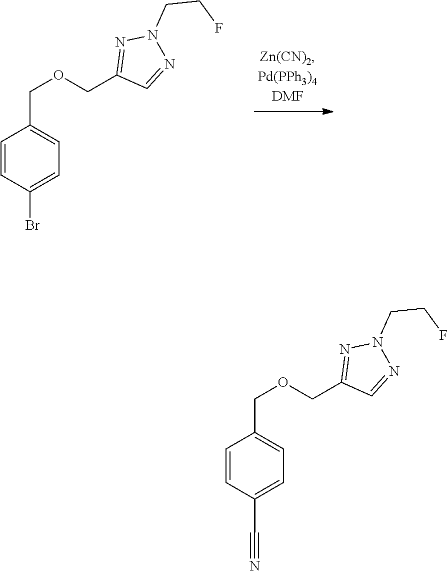 [SMILES: FCCn1ncc(COCc2ccc(Br)cc2)n1 ; N#Cc1ccc(COCc2cnn(CCF)n2)cc1]

To a solution of 4-(((4-bromobenzyl)oxy)methyl)-2-(2-fluoroethyl)-2H-1,2,3-triazole (700 mg, 2.23 mmol) in DMF (10 mL) under a nitrogen atmosphere was added Pd(PPh₃)₄ (514 mg, 445 umol) and Zn(CN)₂ (523 mg, 4.46 mmol) and the reaction mixture was stirred at 100° C. for 12 h. The mixture was diluted with ethyl acetate (50 mL) and H₂O (50 mL). The mixture was filtered over celite and the organic layer was separated, washed with brine (50 mL), dried over Na₂SO₄, filtered and concentrated. The residue was purified by prep-HPLCcolumn: Phenomenex luna C18 150*40 mm*15 um; mobile phase: [water(0.225% FA)-ACN]; B %: 20%-50%, 9 min to give the desired compound. The product compound 4B (300 mg, 7% yield) was obtained as yellow solid. LCMS: m/z=261.1 [M+H]⁺. ¹HNMR: (400 MHz, CDCl₃) δ 7.66 (d, J=1.6 Hz, 1H), 7.61-7.55 (m, 2H), 7.50 (br d, J=4.8 Hz, 2H), 4.98 (t, J=4.8 Hz, 1H), 4.86 (t, J=4.8 Hz, 1H), 4.76 (t, J=4.8 Hz, 1H), 4.72-4.68 (m, 2H), 4.72-4.68 (m, 1H), 4.65 (s, 2H).

### Preparation of 3-(4-(((2-(2-fluoroethyl)-2H-1,2,3-triazol-4-yl)methoxy)methyl)phenyl)-1,2,4,5-tetrazine (Compound 3′)

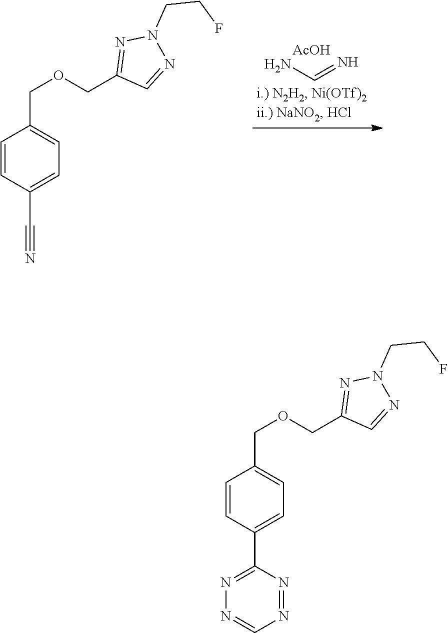

A 40 mL sealed tube was charged with a solution of 4-(((2-(2-fluoroethyl)-2H-1,2,3-triazol-4-yl)methoxy)methyl)benzonitrile (300 mg, 1.15 mmol), methanimidamide acetate (1.20 g, 11.5 mmol), Ni(OTf)₂ (205 mg, 576 umol) in N₂H₄.H₂O (2.94 g, 57.6 mmol). The mixture was stirred at 35° C. for 12 h. A solution of NaNO₂ (1.59 g, 23.0 mmol, 20.0 eq) in H₂O (5 mL) was added into the mixture at 5° C. and followed by slow addition of 1 M HCl during which the solution turned bright red in color and gas evolved, addition of 1 M HCl continued until gas evolution ceased and the pH value is 3. The mixture was extracted with EtOAc (100 mL×3), the organic layer was washed with brine (100 mL×2), dried over Na₂SO₄, filtered and concentrated. The residue was purified by prep. HPLC column: Phenomenex Synergi C18 150*25*10 um; mobile phase: [water(0.225% FA)-ACN]; B %: 38%-68%, 10 min to give the desired compound. The title compound (18.0 mg, 5% yield) was obtained as red solid. LCMS: m/z=356.2 [M+H]⁺. ¹H NMR: (400 MHz, CDCl₃) δ 10.23 (s, 1H), 8.63 (d, J=8.4 Hz, 2H), 7.69 (s, 1H), 7.61 (d, J=8.4 Hz, 2H), 4.98 (t, J=4.8 Hz, 1H), 4.88-4.85 (m, 1H), 4.78-4.75 (m, 2H), 4.72 (s, 2H), 4.72-4.68 (m, 2H).

### Example 5: Synthesis of N-(4-(1,2,4,5-tetrazin-3-yl)benzyl)-1-(4-fluorophenyl)methanamine (Compound 4′)

### Preparation of tert-Butyl (4-(1,2,4,5-tetrazin-3-yl)benzyl)(methyl)carbamate

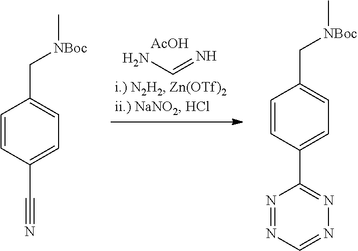

tert-butyl (4-cyanobenzyl)(methyl)carbamate (1.90 g, 7.71 mmol), methanimidamide acetate (8.03 g, 77.1 mmol) and DMF (9.00 mL) were charged into a one-necked flask. To this solution was added Zn(OTf)₂.H₂O (1.47 g, 3.86 mmol) and NH₂NH₂.H₂O (19.3 g, 386 mmol, 18.8 mL). The mixture was stirred at 30° C. for 12 h under N₂ atmosphere. The reaction solution was cooled to 20° C. NaNO₂ (10.7 g, 154 mmol) in 100 mL of water was slowly added to the solution at 0° C. and followed by slow addition of 1M HCl during which the solution turned bright red in color and gas evolved at 0° C. Addition of 1M HCl continued until gas evolution ceased and the pH value is 3. The mixture was extracted with EtOAc (500 mL). The organic phase was washed with brine (300 mL), dried over Na₂SO₄, filtered and concentrated. The residue was purified by silica gel column chromatography (Petroleum ether:Ethyl acetate=20:1) to give a purple solid. The solid was purified by prep-HPLC (column: Phenomenex luna C18 150*40 mm*15 um; mobile phase: [water(0.1% TFA)-ACN]; B %: 48%-68%, 10 min) to give 300 mg crude residue, which was purified further by prep-HPLC (column: Phenomenex Gemini-NX C18 75*30 mm*3 um; mobile phase: [water(0.1% TFA)-ACN]; B %: 55%-65%, 7 min) and the elution was extracted with ethyl acetate (50 mL) washed with brine (50 mL), dried over Na₂SO₄, filtered and concentrated to give the title compound (230 mg, 10% yield) as purple solid. ¹H NMR: (400 MHz, CDCl₃) δ 10.22 (s, 1H), 8.61 (d, J=8.40 Hz, 2H), 7.47 (br d, J=7.20 Hz, 2H), 4.55 (br s, 2H), 3.01-2.82 (m, 3H), 1.50 (m, 9H).

### Preparation of 1-(4-(1,2,4,5-tetrazin-3-yl)phenyl)-N-methylmethanamine

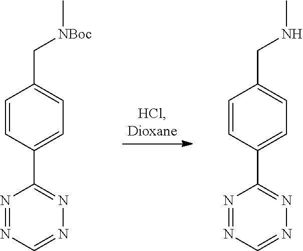 [SMILES: CN(Cc1ccc(-c2nncnn2)cc1)C(=O)OC(C)(C)C ; CNCc1ccc(-c2nncnn2)cc1]

tert-butyl (4-(1,2,4,5-tetrazin-3-yl)benzyl)(methyl)carbamate (230 mg, 763 umol) and dioxane (5 mL) were charged into a one-necked flask. To this solution was added HCl/dioxane (4 M, 5 mL). The reaction was stirred at 20° C. for 1 h. The mixture was concentrated to give the title compound (100 mg, 55% yield, HCl) as red solid. ¹HNMR: (400 MHz, DMSO-d6) δ 10.64 (s, 1H), 9.15 (br s, 2H), 8.56 (d, J=8.40 Hz, 2H), 7.80 (d, J=8.40 Hz, 2H), 4.27 (t, J=5.60 Hz, 2H), 2.61 (t, J=5.20 Hz, 3H).

1-(4-(1,2,4,5-tetrazin-3-yl)phenyl)-N-methylmethanamine can be used to prepare compounds with a fluorophenyl group.

### Preparation of N-(4-(1,2,4,5-tetrazin-3-yl)benzyl)-1-(4-fluorophenyl)methanamine (Compound 4′)

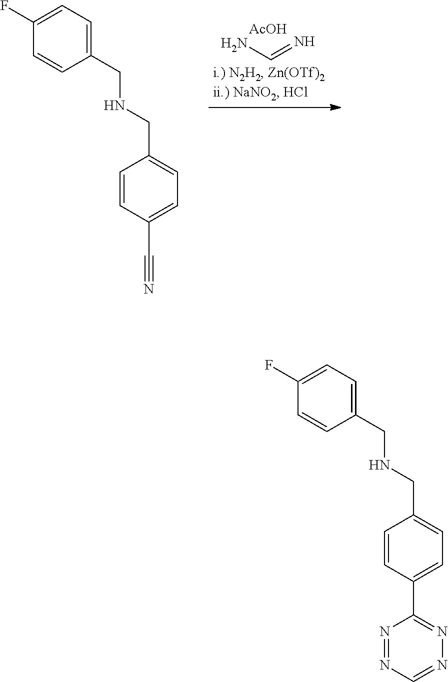

4-(((4-fluorobenzyl)amino)methyl)benzonitrile (3.00 g, 12.49 mmol), methanimidamide acetate (13.0 g, 125 mmol) and DMF (15.0 mL) were charged into a one-necked flask. To this solution was added Zn(OTf)₂.H₂O (2.38 g, 6.24 mmol) and NH₂NH₂.H₂O (31.3 g, 624 mmol, 30.3 mL). The mixture was stirred at 30° C. for 36 h under N₂ atmosphere. The reaction solution was cooled to 20° C. NaNO₂ (17.2 g, 250 mmol) in 100 mL of water was slowly added to the solution at 0° C. and followed by slow addition of 1M HCl during which the solution turned bright red in color and gas evolved at 0° C. Addition of 1M HCl continued until gas evolution ceased and the pH value is 3. The mixture was extracted with DCM:MeOH=10:1 500 mL×3. The organic phase was dried over Na₂SO₄, filtered and concentrated. The residue was purified by prep-HPLC (column: Phenomenex luna C18 250*80 mm*10 um; mobile phase: [water(0.225% FA)-ACN]; B %: 10ACN %-40ACN %, 12 min) to give the title compound (50 mg, 1% yield) as red solid. ¹HNMR: (400 MHz, DMSO-d6) δ 10.64 (s, 1H), 9.29 (br s, 2H), 8.58 (d, J=8.40 Hz, 2H), 7.79 (d, J=8.40 Hz, 2H), 7.62-7.53 (m, 2H), 7.37-7.28 (m, 2H), 4.37-4.24 (m, 4H).

### Example 6: Synthesis of N-(4-(1,2,4,5-tetrazin-3-yl)benzyl)-1-(4-fluorophenyl)-N-methylmethanamine (Compound 5′)

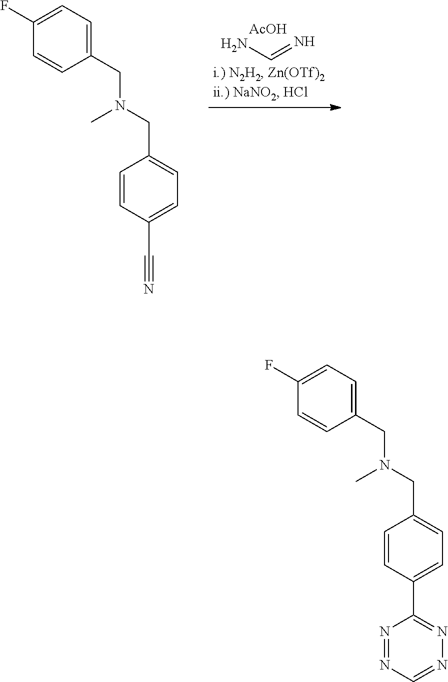

4-(((4-fluorobenzyl)(methyl)amino)methyl)benzonitrile (3.00 g, 11.8 mmol, 1.00 eq), methanimidamide acetate (12.3 g, 118 mmol) and DMF (15.0 mL) were charged into a one-necked flask. To this solution was added Zn(OTf)₂.H₂O (2.25 g, 5.90 mmol) and NH₂NH₂.H₂O (29.5 g, 590 mmol, 28.7 mL). The mixture was stirred at 30° C. for 24 h under N₂ atmosphere. The reaction solution was cooled to room temperature. NaNO₂ (16.3 g, 236 mmol) in 100 mL of water was slowly added to the solution at 0° C. and followed by slow addition of 1M HCl during which the solution turned bright red in color and gas evolved at 0° C. Addition of 1M HCl continued until gas evolution ceased and the pH value is 3. The mixture was extracted with DCM:MeOH (10:1, 500 mL×4). The organic phase was dried over Na₂SO₄, filtered and concentrated. The residue was purified by prep-HPLC(column: Phenomenex luna C18 (250*70 mm, 10 um); mobile phase: [water(0.225% FA)-ACN]; B %: 15%-40%, 20 min) to give crude 500 mg, which was purified by prep-HPLC(column: Phenomenex luna C18 150*25 mm*10 um; mobile phase: [water(0.1% TFA)-ACN]; B %: 11%-41%, 10 min) to give a rose red solid 200 mg. Then the solid was purified by prep-HPLC(column: Waters Xbridge 150*25 mm*Sum; mobile phase: [water(10 mM NH4HCO3)-ACN]; B %: 50%-80%, 10 min) and the elution was acified with conC. HCl to pH=3, freeze-dried to give pure compound, which was purified by prep-HPLC(column: Phenomenex Synergi C18 150*25*10 um; mobile phase: [water(0.05% HCl)-ACN]; B %: 13%-43%, 10 min). The title compound (56 mg, 1% yield, HCl) was obtained as a red solid. ¹HNMR: (400 MHz, Methanol-d4) δ 10.40 (s, 1H), 8.77-8.69 (m, 2H), 7.81 (d, J=8.40 Hz, 2H), 7.65-7.56 (m, 2H), 7.26 (t, J=8.80 Hz, 2H), 2.79 (s, 3H).

### Example 7. Synthesis of N-(4-(1,2,4,5-tetrazin-3-yl)benzyl)-4-fluorobenzamide (Compound 6′)

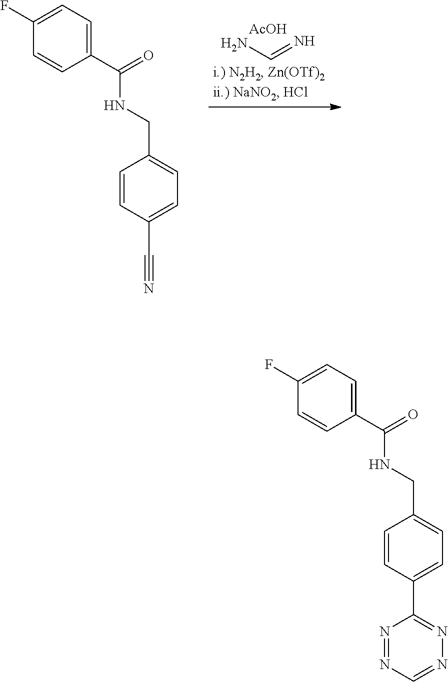

N-(4-cyanobenzyl)-4-fluorobenzamide (3.00 g, 11.8 mmol), methanimidamide acetate (12.3 g, 118 mmol) and DMF (14.0 mL) were charged into a one-necked flask. To this solution was added Zn(OTf)₂.H₂O (2.25 g, 5.90 mmol) and NH₂NH₂.H₂O (29.5 g, 590 mmol, 28.7 mL). The mixture was stirred at 30° C. for 12 h under N₂ atmosphere. The reaction solution was cooled to room temperature. NaNO₂ (16.3 g, 236 mmol) in 100 mL of water was slowly added to the solution at 0° C. and followed by slow addition of 1M HCl during which the solution turned bright red in color and gas evolved at 0° C. Addition of 1M HCl continued until gas evolution ceased and the pH value is 3. The mixture was extracted with EtOAc (500 mL). The organic phase was washed with brine (300 mL), dried over Na₂SO₄, filtered and concentrated. The residue was purified by silica gel column chromatography (Petroleum ether:Ethyl acetate=5:1-1:1) to give a purple solid 2.0 g. The material was purified by prep-HPLC(column: Phenomenex luna C18 150*40 mm*15 um; mobile phase: [water(0.1% TFA)-ACN]; B %: 32%-52%, 10 min) to give a purple solid, which was purified by prep-HPLC again (column: Phenomenex Synergi C18 150*25*10 um; mobile phase: [water(0.225% FA)-ACN]; B %: 27%-57%, 10 min) to give compound 250 mg as purple solid. Then the compound was purified by prep-HPLC (column: Shim-pack C18 150*25*10 um; mobile phase: [water(0.225% FA)-ACN]; B %: 33%-57%, 11 min) and freeze-dried to give the title compound (50 mg, 1% yield) as purple solid. ¹HNMR: (400 MHz, CDCl₃) δ 10.23 (s, 1H), 8.63 (d, J=8.40 Hz, 2H), 7.92-7.79 (m, 2H), 7.61 (d, J=8.40 Hz, 2H), 7.15 (t, J=8.40 Hz, 2H), 6.50 (br s, 1H), 4.79 (d, J=6.00 Hz, 2H).

### Example 8: Synthesis of N-(4-(1,2,4,5-tetrazin-3-yl)benzyl)-4-fluoro-N-methylbenzamide (Compound 7′)

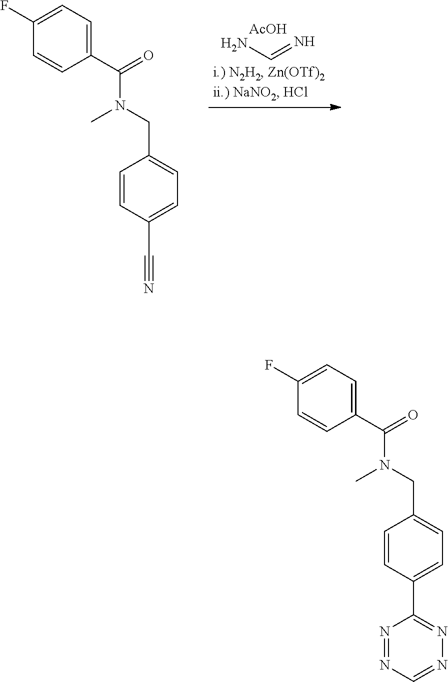

N-(4-cyanobenzyl)-4-fluoro-N-methylbenzamide (2.50 g, 9.32 mmol), methanimidamide acetate (9.70 g, 93.2 mmol) and DMF (11.0 mL) were charged into a one-necked flask. To this solution was added Zn(OTf)₂.H₂O (1.78 g, 4.66 mmol, 0.50 eq) and NH₂NH₂.H₂O (23.3 g, 466 mmol, 22.6 mL). The mixture was stirred at 30° C. for 12 h under N₂ atmosphere. The reaction solution was cooled to 20° C. NaNO₂ (12.9 g, 186 mmol) in 100 mL of water was slowly added to the solution at 0° C. and followed by slow addition of 1M HCl during which the solution turned bright red in color and gas evolved at 0° C. Addition of 1M HCl continued until gas evolution ceased and the pH value is 3. The mixture was extracted with EtOAc (500 mL). The organic phase was washed with brine (300 mL), dried over Na₂SO₄, filtered and concentrated. The residue was purified prep-HPLC(column: Phenomenex luna C18 150*40 mm*15 um; mobile phase: [water(0.1% TFA)-ACN]; B %: 40%-50%, 10 min) to give a purple solid 300 mg, which was purified by prep-HPLC(column: Phenomenex Gemini-NX C18 75*30 mm*3 um; mobile phase: [water(0.1% TFA)-ACN]; B %: 40%-50%, 7 min) again to give the title compound (50 mg, 2% yield) as a red solid. ¹HNMR: (400 MHz, CDCl₃) δ 10.24 (s, 1H), 8.65 (d, J=8.40 Hz, 2H), 7.72-7.36 (m, 4H), 7.12 (br s, 2H), 4.95-4.58 (m, 2H), 3.18-2.92 (m, 3H).

### Example 9: Synthesis of 3-(4-((2-fluoroethoxy)methyl)phenyl)-1,2,4,5-tetrazine (Compound 8′)

### Preparation of 4-((2-fluoroethoxy)methyl)benzonitrile

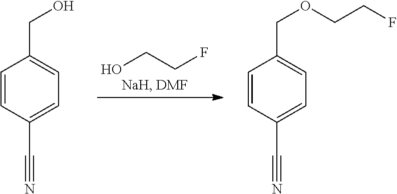

2-Fluoroethanol (718 mg, 11.2 mmol) was added slowly to a suspension of NaH (816 mg, 20.4 mmol, 60.0% purity) in DMF (5.0 mL) at 0° C. then warmed to 25° C. After the hydrogen evolution ceased, 4-(hydroxymethyl)benzonitrile (2.00 g, 10.2 mmol) was added dropwise. The reaction mixture was stirred for 4 h at 25° C. The residue was purified by column chromatography (SiO₂, Petroleum ether:Ethyl acetate=20:1 to 10:1) to afford title compound (1.00 g, 55% yield) as a white solid. ¹HNMR: (400 MHz, CDCl₃) δ 7.56 (d, J=8.25 Hz, 2H), 7.39 (d, J=8.00 Hz, 2H), 4.58-4.62 (m, 1H), 4.58 (s, 2H), 4.46-4.50 (m, 1H), 3.71-3.76 (m, 1H), 3.64-3.68 (m, 1H).

### Preparation of 3-(4-((2-fluoroethoxy)methyl)phenyl)-1,2,4,5-tetrazine (Compound 8′)

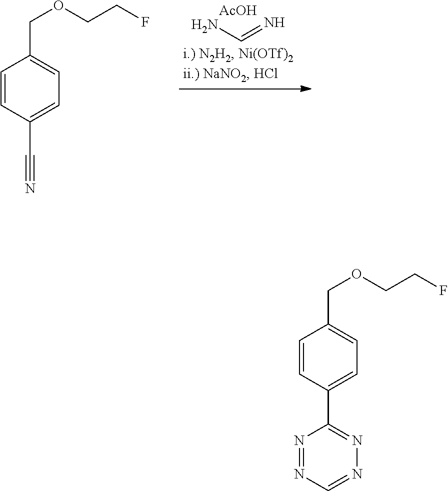

A mixture of 4-((2-fluoroethoxy)methyl)benzonitrile (0.50 g, 2.79 mmol), methanimidamide acetate (2.90 g, 27.9 mmol), N₂H₄.H₂O (7.13 g, 139 mmol, 6.92 mL) and Ni(OTf)₂ (497 mg, 1.40 mmol) was stirred at 25° C. for 12 h. NaNO₂ (3.85 g, 55.8 mmol) in H₂O (5.00 mL) was slowly added to the solution and followed by slow addition of 1M HCl during which the solution turned bright red in color and gas evolved. Addition of 1M HCl continued until gas evolution ceased and the pH value is 3. The product was purified by prep-HPLC (column: Phenomenex Synergi Max-RP 250*50 mm*10 um; mobile phase: [water(0.1% TFA)-ACN]; B %: 15%-50%, 27 MIN; 30% min) to afford the title compound (70.0 mg, 11% yield) was obtained as a red solid. LCMS: (ESI) m z 235.0 [M+H]. ¹HNMR: (400 MHz, CDCl₃) δ 10.23 (s, 1H), 8.64 (d, J=8.38 Hz, 2H), 7.62 (d, J=8.38 Hz, 2H), 4.74 (s, 2H), 4.70-4.73 (m, 1H), 4.57-4.61 (m, 1H), 3.84-3.88 (m, 1H), 3.77-3.80 (m, 1H).

### Example 10: Synthesis of Malat1 ASO-TCO

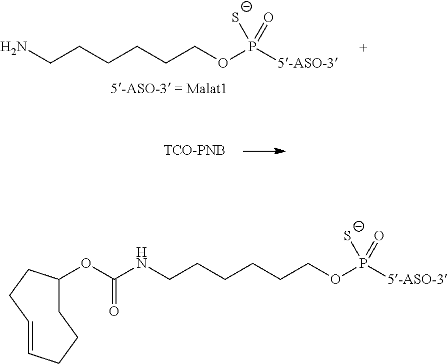

5′-Hexyl amino oligo was dissolved in borate buffer (100 mM, pH=8.5) to a concentration of 50-100 mg/ml. To this solution was added of TCO-PNB (4 equiv, Click Chem Tools: 1192) dissolved in an equal volume of DMF. (TCO-NHS (CCT 1016) can be substituted for TCO-PNB, and 5-6 eq dissolved in an equal volume of DMF (i.e., final solution is 1:1 Organic:Aq) is employed.) The mixture was stirred at room temperature for 30 min. The mixture was diluted with water (8 mL) and filtered through a 0.2 micron PTFE syringe filter. The material was purified by SAX-HPLC using a linier gradient (A: 100 mM NH₄OAc/30% MeCN B: 100 mM NH₄OAc/1.5M NaBr/30% MeCN). The product fractions are pooled and the MeCN removed using a rotovap (bath temp >25° C.). The aqueous solution was loaded onto a 5 g C-18 SPE column, and subsequently washed with 1 m NaCl, followed by water, and the desalted product was eluted with 30 ml 1:1 MeCN/H₂O. The resultant solution was concentrated by lyophilization to give a white powder.

### Example 11: In Vitro Characterization

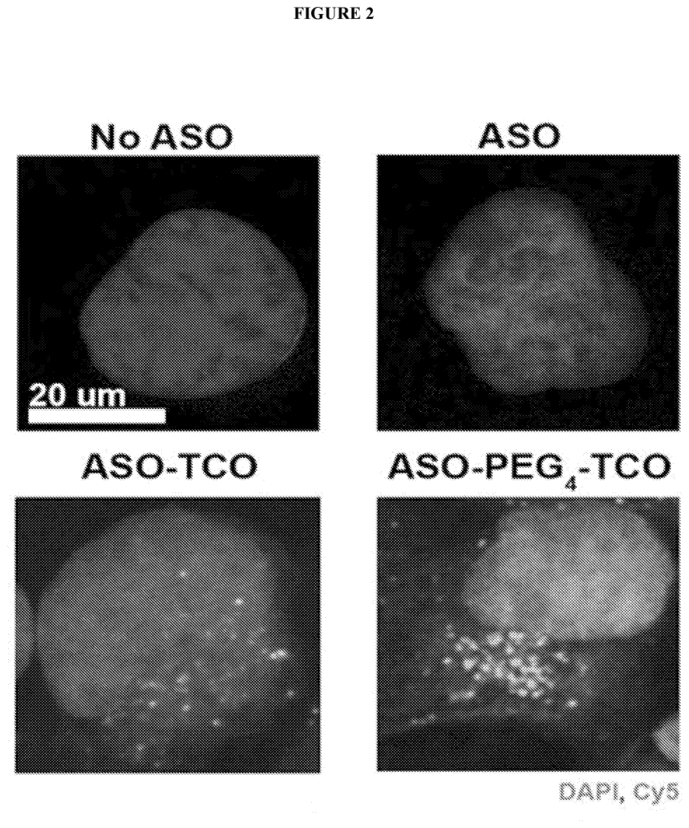

As a model for antisense oligonucleotide (ASO) pretargeting, a phosphorothioate backbone Malat-1 ASO was conjugated on its 5′ end with a bifunctional TCO-linker (ASO-TCO). The Malat-1 ASO is: 5′-GCCAGGCTGGTTATGACTCA-3′ (SEQ ID NO:1) wherein the bolded nucleobases are 2′-MOE; the nucleobases in regular font are DNA (i.e., sugar 2′positions are H); the underlined residues have phosphodiester linkage; the others have phosphorothioate backbone. Next, ASO-TCO uptake in cells was visualized by confocal microscopy through incubation with a tetrazine (Tz)-Cy5 fluorophore. Specifically, HeLa cells were incubated with either a Malat1 ASO, Malat1 ASO-trans cyclooctene (TCO), or Malat1 ASO-PEG₄-TCO for 24 hours at 37° C. Cells were then fixed in 4% paraformaldehyde (PFA), permeabilized with 0.1% Triton X-100 in phosphate buffered saline (PBS) and stained using tetrazine-Cy5. Tetrazine reacted covalently with TCO on the ASO conjugates but demonstrated no binding in the presence of ASO alone (FIG. 2). These data show that ASO-TCO conjugates are taken up into the cell by endosomal pathways (data not shown) and remain reactive to tetrazine moieties.

As pretargeting is a 2-component system, an ¹⁸F-Tz was developed to be CNS-penetrant based on cnsPET-MPO modeling (Zhang, L., et al. (2018). J Med Chem 61(8): 3296-3308). The non-radioactive analogue of the ligand was tested in vitro and displayed favorable brain and plasma protein binding (51% and 69% unbound respectively) and predicted to be brain penetrant based on efflux ratios in MDCK-P-gp (1.71) and MDCK-BCRP (0.51) cell models of permeability. Radiosynthesis of the ¹⁸F-Tz ligand resulted in high radiochemical purity (RCP) (>99%) and molar activity (4200 Ci/mmol). Subsequent evaluation of the radiotracer in naïve mice by dynamic PET/CT demonstrated brain uptake (1.7±0.9% ID by 10 min P.I.) and clearance, suggesting effective use as a CNS pretargeted imaging agent.

### Example 12: In Vivo Imaging

The two components for the pretargeting strategy were then brought together for in vivo pretargeted imaging studies (FIG. 3).

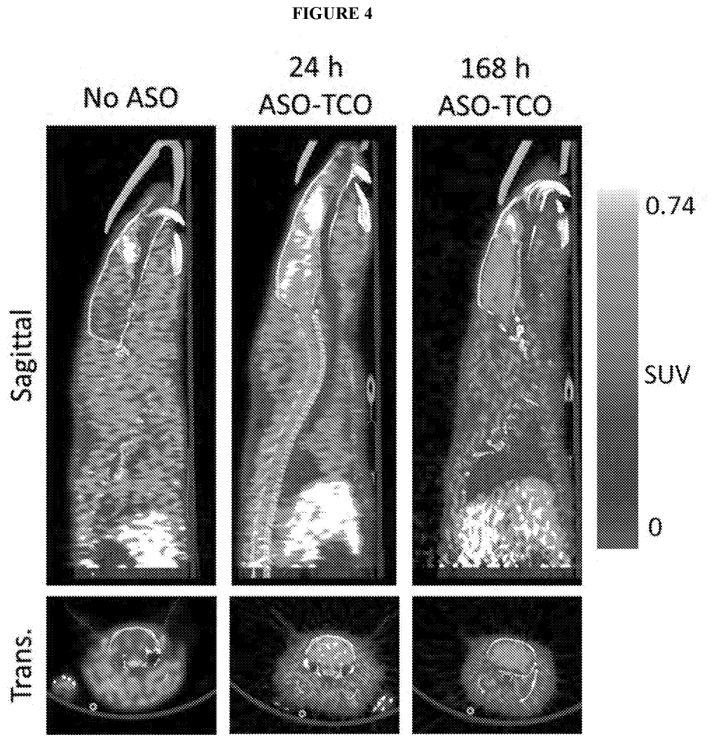

Rats were administered Malat1 ASO-TCO intrathecally (I.T.) and 24 hours later were given ¹⁸F-Tz intravenously (I.V.). They were then imaged 75-90 p.i. by PET/CT. Images show specific uptake of tracer in the brain and spinal cord in rats treated with ASO-TCO (FIG. 4).

The same uptake is not seen in rats that received only the ¹⁸F-Tz radiotracer. Brain-to-heart and spine-to-heart ratios (center) derived from ROIs were significantly higher in rats (P<0.005) treated with ASO-TCO than control rats that received only tracer.

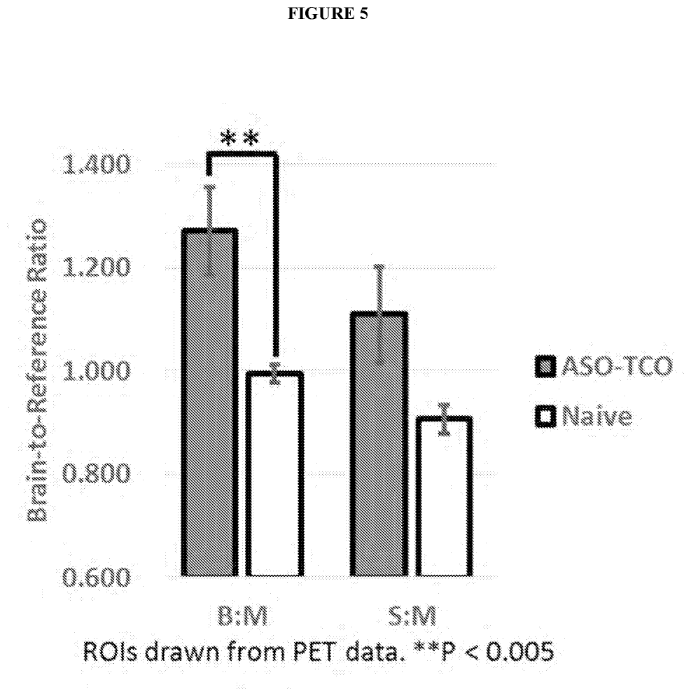

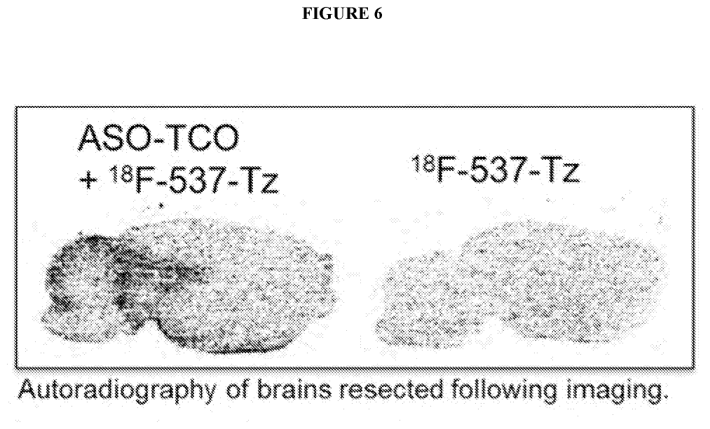

Immediately following imaging, brains were resected, sectioned, and exposed to phosphor plate (FIG. 5). Autoradiography demonstrated a pattern of distribution characteristic of intrathecally administered ASOs, while control brains did not (FIG. 6). Autoradiography studies using tissue treated in vivo with ASO-TCO demonstrated that radiotracer signal could be blocked with an excess of cold tetrazine ligand. This suggests that the observed PET signal is due to the in vivo click ligation between the TCO-labeled ASO and the Tz-labeled radioligand.

As current ASO imaging relies on direct labeling with longer-lived radioisotopes, the present techniques allow for the development of new ASO-based therapies by elucidating long-term temporal distributions in vivo while maintaining a low radiation exposure to the patient.

### Example 13: Dynamic Scans of Malat1 ASO-TCO Rats Using ¹⁸F-537-Tz

¹⁸F-537-Tz was produced by a 3-step radiosynthesis and reformulated in 10% EtOH: 90% 0.9% saline solution. Typically, the total synthesis procedure was accomplished in 120 min from end of bombardment (EOB). Up to 500 MBq of ¹⁸F-537-Tz was synthesized with a molar activity (Aₘ) of 144±42 GBq/μmol at EOS and in high radiochemical purity (>97%). Identity and purity (chemical and radiochemical) of ¹⁸F-537-Tz doses were determined by HPLC analysis. The average activity of ¹⁸F-537-Tz injected was 11.09±2.36 MBq and the average mass of ¹⁸F-537-Tz injected was 0.04±0.02 μg.

Dynamic PET scans were performed with arterial sampling to determine parent fraction of the tracer ¹⁸F-537-Tz in baseline and pretargeted scans. Baseline imaging with a comparison to a homologous block with non-radioactive compound ¹⁹F-537-Tz at 1 mg/kg was performed on a single rat, where non-radioactive compound was administered i.v. 5 min before injection of tracer. Next, three additional rats were imaged at baseline with no ASO-TCO administered. A final three rats received pretargeted scans and were dosed intrathecally with 0.56 mg Malat1 ASO-TCO in 30 μL saline followed by a 40 μL saline flush. 24 h after injection of the ASO, ¹⁸F-537-Tz was injected intravenously and dynamic PET imaging performed.

A metabolite analysis method was developed to enable the tracer kinetic modelling using metabolite corrected plasma activity as an input function. The plasma radioactivity extraction efficiency for all samples was determined and was satisfactory using 1:1 plasma:ACN. The recovery from the HPLC column of the injected radioactivity was determined for each plasma extract injected. Good recovery of the injected radioactivity from the HPLC column was obtained for each plasma extract injection. For the 3 baseline scans, the parent compound fraction of the total activity found in plasma was 86-90% at 5 min, and this fraction decreased to 66-74% at 60 min after ¹⁸F-537-Tz injection. The parent fraction was reduced slightly in the rat dosed with ¹⁸F-537-Tz at 1 mg/kg; at 5 min after tracer injection, the parent compound fraction of the total activity found in plasma was 82% decreasing to 62% at 60 min. The parent fraction was also comparable with the rats dosed with ASO-TCO by i.t; at 5 min after tracer injection, the parent compound fraction of the total activity found in plasma was 88%-89% decreasing to 66%-69% at 60 min.

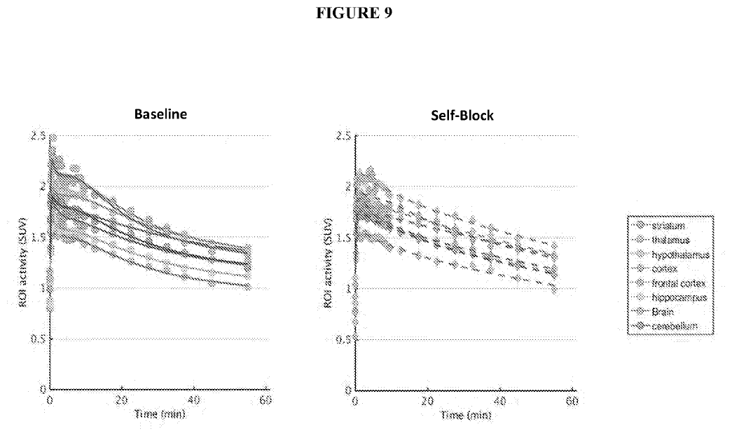

Time-activity-curves (TAC) from 0 to 60 min showing the brain subregion distribution of ¹⁸F-537-Tz in rat 2 at baseline and following iv administration of 1 mg/kg of unlabeled ¹⁸F-537-Tz are illustrated in FIG. 9. The uptake of radioactivity was observed in the brain of both the baseline and post dose scans. The TACs indicated that ¹⁸F-537-Tz readily entered the brain with an initial peak uptake and a tissue washout that appears conducive to a robust kinetic parameters estimation. The average brain regional SUV from 40-60 min of the scans were similar in the baseline and homologous block scans (Table 1).

TABLE 1
Comparison of baseline vs. homologous block (postdose) SUV from 40-60 min p.i. of tracer.

| | | | | | | | Frontal | | | Plasma |
|---|---|---|---|---|---|---|---|---|---|---|
| Subject | Scan | Striatum | Thalamus | Hypothalamus | Cerebellum | Cortex | Cortex | Hippocampus | Brain | (40-60 min) |
| 2 | Baseline | 1.42 | 1.46 | 1.45 | 1.38 | 1.07 | 1.16 | 1.28 | 1.29 | 1.12 |
| 2 | Postdose | 1.40 | 1.52 | 1.43 | 1.38 | 1.10 | 1.24 | 1.27 | 1.22 | 1.45 |

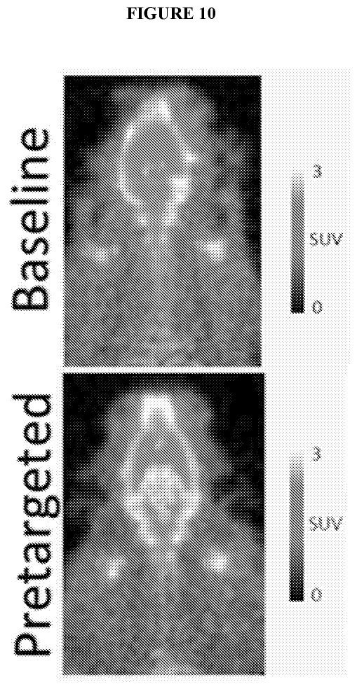

PET/CT images with summed radioactivity from 0 to 60 min showing the brain distribution of ¹⁸F-537-Tz in the 4 rats scanned at baseline and the 3 rats scanned 24 h after i.t. administration of ASO-TCO are illustrated in FIG. 10. There was an increase in brain uptake of radioactivity observed in all the ASO-TCO dosed rats. There was an increase of ca. 25% in the SUV (30-60 min) in the brain of the rats pretreated with ASO-TCO as compared to the rats scanned at baseline (Tables 2 and 3).

TABLE 2
Comparison of baseline vs. pretreated SUV in brain subregions from 40-60 min p.i. of tracer.

| | | | | | | Frontal | | Whole | |
|---|---|---|---|---|---|---|---|---|---|
| Group | Striatum | Thalamus | Hypothalamus | Cerebellum | Cortex | Cortex | Hippocampus | Brain | Plasma |
| Baseline | 1.52 | 1.64 | 1.62 | 1.52 | 1.25 | 1.33 | 1.43 | 1.45 | 1.22 |
| Std. Dev | 0.08 | 0.12 | 0.13 | 0.14 | 0.13 | 0.12 | 0.1 | 0.12 | 0.09 |
| Pretargeted | 1.77 | 1.95 | 1.89 | 1.9 | 1.66 | 1.66 | 1.85 | 1.79 | 1.18 |
| Std. Dev. | 0.09 | 0.12 | 0.08 | 0.14 | 0.12 | 0.07 | 0.17 | 0.1 | 0.10 |
| % Change | 16 | 19 | 17 | 25 | 33 | 25 | 29 | 23 | |

TABLE 3
Comparison of baseline vs. pretreated SUV in brain subregions from 40-60 min p.i. of tracer normalized to plasma.

| | | | | | | Frontal | | Whole |
|---|---|---|---|---|---|---|---|---|
| Group | Striatum | Thalamus | Hypothalamus | Cerebellum | Cortex | Cortex | Hippocampus | Brain |
| Baseline | 1.27 | 1.37 | 1.36 | 1.27 | 1.05 | 1.11 | 1.2 | 1.21 |
| Std. Dev | 0.05 | 0.09 | 0.09 | 0.06 | 0.07 | 0.06 | 0.07 | 0.06 |
| Pretargeted | 1.47 | 1.61 | 1.63 | 1.67 | 1.42 | 1.43 | 1.61 | 1.53 |
| Std. Dev. | 0.17 | 0.22 | 0.19 | 0.26 | 0.22 | 0.17 | 0.29 | 0.21 |
| % Change | 16 | 18 | 20 | 31 | 35 | 29 | 34 | 26 |

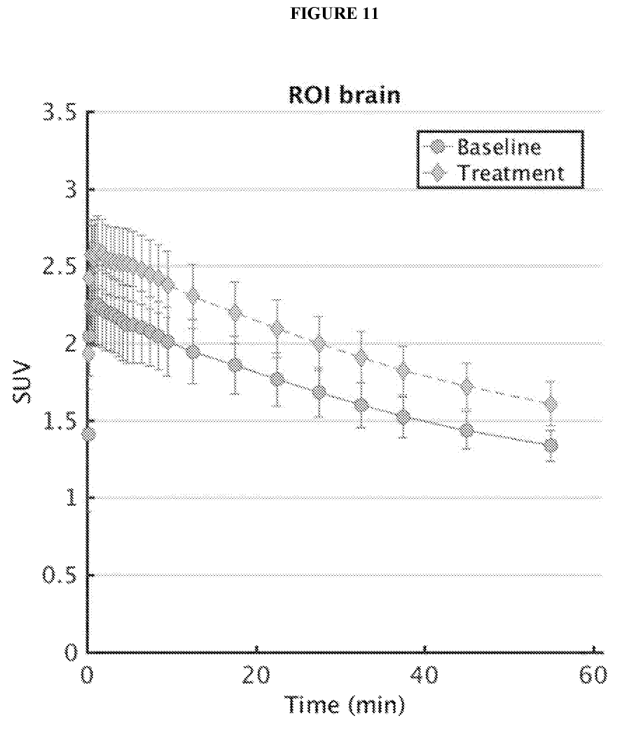

The TACs of uptake in the whole brain ROI of the rats in the baseline and post dose groups were plotted on the same graph for comparison in FIG. 11.

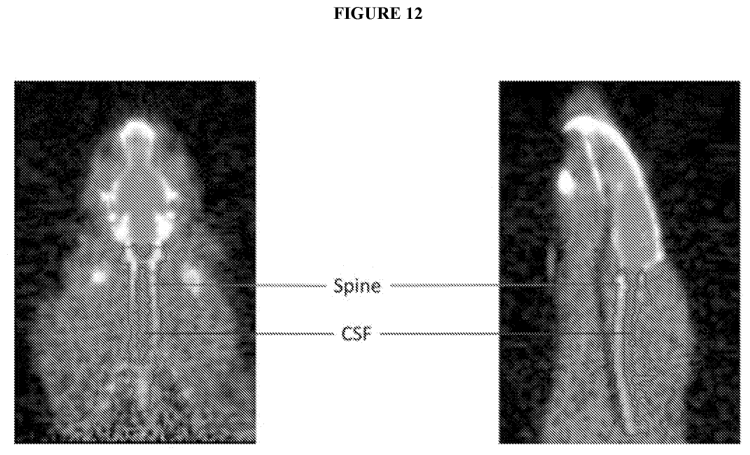

The static PET/CT images of ¹⁸F-537-Tz uptake in the upper rat spine are illustrated in FIG. 12. Spine ROI and CSF ROI were defined to generate SUV measures at 30-60 min (spine ROI was a segmentation of the vertebrae (used to aid segmentation of the CSF) and the CSF ROI was everything within the spine). There was a 10% and 15% increase in the radioactivity uptake in the spine and CSF when comparing the baseline and post ASO-TCO dosed rats (Table 4).

TABLE 4
Radioactivity uptake in the rat spine and CSF following i.t.
dosing of ASO-TCO (ca. 70-80 min after tracer injection)

| Group | Spine | CSF |
|---|---|---|
| Baseline | 0.95 | 1.18 |
| Std. Dev | 0.06 | 0.10 |
| Pretargeted | 1.04 | 1.37 |
| Std. Dev. | 0.1 | 0.06 |
| % Change | 9 | 16 |

### Conclusion

¹⁸F-537-Tz readily entered the brain with highest uptake in the thalamus/hypothalamus and lowest in the cortical regions. Intrathecal administration of ASO-TCO 24 h before PET scanning resulted in ˜25% in SUV (30-60 min) in the brain of the rats pretreated with ASO-TCO as compared to the rats scanned at baseline.

### Example 14: Pretargeted PET Imaging in NHP

The pretargeting PET tracer ¹⁸F-537-Tz was tested in a cynomolgus monkey, including baseline scans and a homologous blocking scan. The monkey was then dosed with Malat1 ASO-TCO and scanned again at 24 h and 168 h. Evidence of tracer binding the ASO in vivo was seen in the brain and spinal cord.

### Methods

Dynamic PET scans (0-120 min) with arterial input function were performed on a female cynomolgous monkey using ¹⁸F-537-Tz (5.7±0.7 mCi, 0.14±0.12 μg) in order to evaluate the tracer's efficacy in non-human primates. Parent fraction of tracer in arterial plasma was determined by extracting tracer using acetonitrile and analysis by radioHPLC. Baseline scans were performed first, measuring tracer kinetics in the brain. Malat1 ASO-TCO (20 mg in 2.4 mL aCSF) was then injected intrathecally under fluoroscopic guidance and ¹⁸F-537-Tz PET/CT imaging performed at 24 h and 168 h under isoflurane anesthesia. Imaging data were co-registered to a cyno brain atlas, and TAC in subregions of interest were derived from them. Static PET/CT scans were acquired in each imaging session, with the field of view (FOV) oriented to capture the spine. This scan took place 130-160 min p.i. of tracer, and an ROI drawn over the spinal cord with muscle as a reference region.

### Results

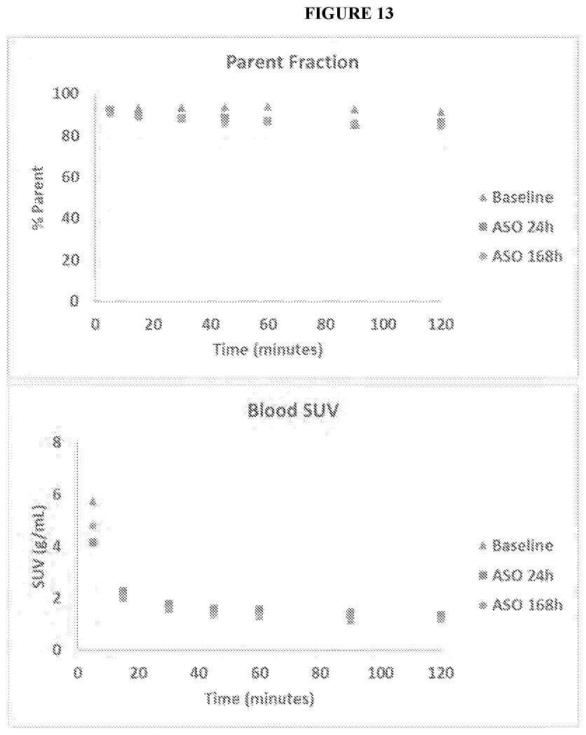

Parent fraction data show the tracer is >80% intact over the course of imaging (FIG. 13). Blood kinetics for the tracer are favorable for imaging, showing uptake and rapid clearance over 30 min.

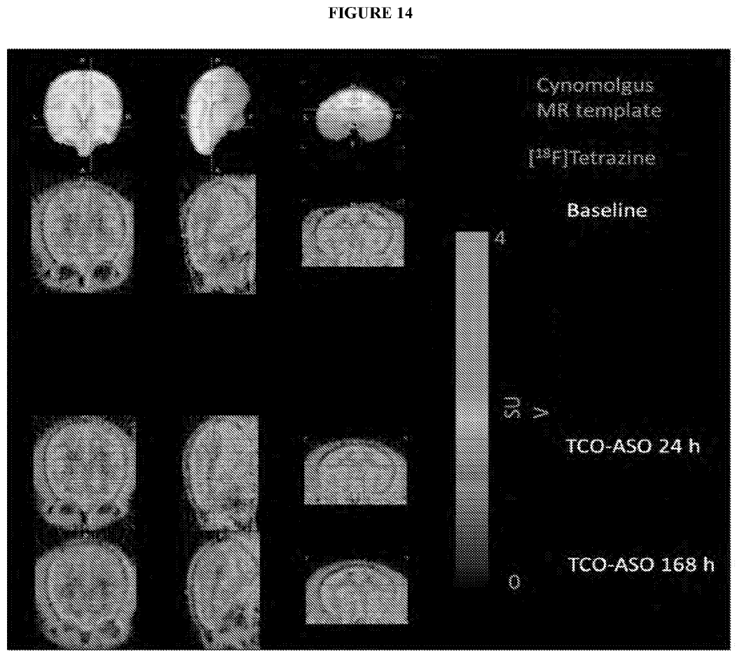

Baseline PET/CT scans show good uptake of the tracer in the brain, with relatively slow clearance in all regions over the 2-hour scan (FIG. 14).

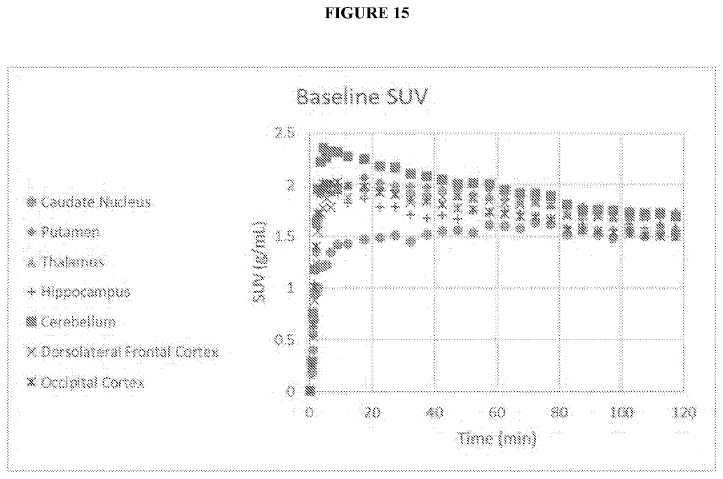

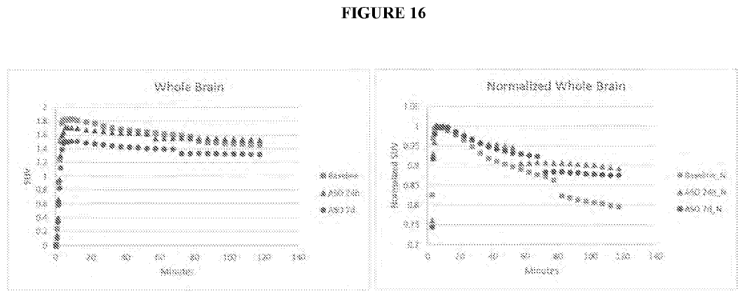

The monkey was injected intrathecally with Malat1 ASO-TCO. After a 24 h interval, ¹⁸F-537-Tz was dosed intravenously and scanned by PET/CT. The monkey was then dosed again with ¹⁸F-537-Tz 7 days post-injection of ASO and scanned by PET/CT. No significant different in SUV uptake is observed between baseline and post-ASO scans (whole brain ROI is presented to represent trends seen in most sub-regions) (FIG. 15). However, when normalizing the TACs, it becomes clear that the tracer in the baseline clears more quickly, while scans post-ASO dosing clear more slowly and appear to plateau by 90 min (FIG. 16). This behavior is because Tz-tracer accumulates in regions as it reacts with the TCO, while unbound tracer clears. By the final timepoint, there is an ˜10% difference in SUV between the baseline and pretargeted scans in the brain.

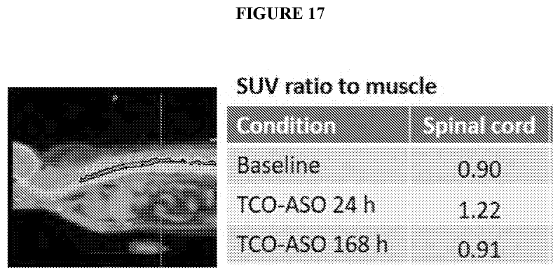

Evidence of tracer undergoing a click reaction and accumulating in tissue can also be seen in the spine. SUV in the spinal cord was normalized to that in muscle, and at the 24 h post-ASO scan the spine shows ˜25% higher signal than in the baseline scan (FIG. 17).

### Conclusion

The tracer ¹⁸F-537-Tz shows excellent uptake in the brain and favorable plasma clearance kinetics. Evidence is shown of specific signal suggesting the tracer binds the Malat1 ASO-TCO in vivo.

### OTHER EMBODIMENTS

While aspects of the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antisense oligonucleotide linked to a trans-cyclooctene.

2. The antisense oligonucleotide of claim 1, wherein the trans-cyclooctene is directly linked to the antisense oligonucleotide.

3. The antisense oligonucleotide of claim 1, wherein the trans-cyclooctene is linked to the antisense oligonucleotide via a linker.

4. The antisense oligonucleotide of claim 3, where the linker is an alkylene linker.

5. The antisense oligonucleotide of any one of claims 1 to 4, wherein the trans-cyclooctene is linked to the antisense oligonucleotide at the 5′-end of the antisense oligonucleotide.

6. The antisense oligonucleotide of any one of claims 1 to 4, wherein the trans-cyclooctene is linked to the antisense oligonucleotide at the 3′-end of the antisense oligonucleotide.

7. The antisense oligonucleotide of any one of claims 4-6, wherein the antisense oligonucleotide is linked to the linker via a phosphorothioate linkage.

8. The antisense oligonucleotide of any one of claims 1 to 5 and 7, wherein the antisense oligonucleotide (ASO) linked to the trans-cyclooctene has a structure of Formula I: 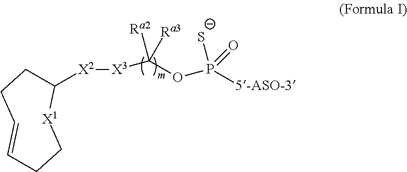
or a salt thereof, wherein:
X¹ is CH₂ or O;
X² is (CH₂)ₜ;
X³ is O, C(O), C(O)O, OC(O), or OC(O)NR^{a1};
R^{a1} is H, C₁₋₆ alkyl, or C₁₋₆ haloalkyl;
R^{a2} and R^{a3} are each independently H or C₁₋₆ alkyl;
t is 0 or 1; and
m is 1, 2, 3, 4, 5, 6, 7, 8, 9, or 10.

9. The antisense oligonucleotide of any one of claims 1 to 4, 6 and 7, wherein the antisense oligonucleotide (ASO) linked to the trans-cyclooctene has a structure of Formula I′: 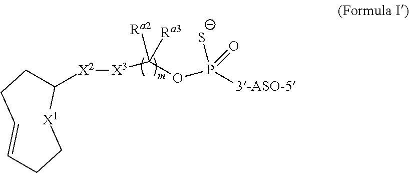
or a salt thereof, wherein:
X¹ is CH₂ or O;
X² is (CH₂)ₜ;
X³ is O, C(O), C(O)O, OC(O), or OC(O)NR^{a1};
R^{a1} is H, C₁₋₆ alkyl, or C₁₋₆ haloalkyl;
R^{a2} and R^{a3} are each independently H or C₁₋₆ alkyl;
t is 0 or 1; and
m is 1, 2, 3, 4, 5, 6, 7, 8, 9, or 10.

10. The antisense oligonucleotide of claim 8 or 9, wherein X¹ is CH₂.

11. The antisense oligonucleotide of claim 8 or 9, wherein X¹ is O.

12. The antisense oligonucleotide of any one of claims 8 to 11, wherein t is 0.

13. The antisense oligonucleotide of any one of claims 8 to 11, wherein t is 1.

14. The antisense oligonucleotide of any one of claims 8 to 13, wherein X³ is OC(O)NR^{a1}.

15. The antisense oligonucleotide of claim 8, wherein the antisense oligonucleotide linked to the trans-cyclooctene has a structure of Formula Ia or Formula Ib: 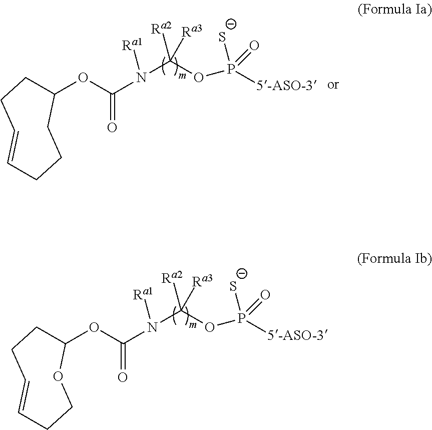
or a salt thereof.

16. The antisense oligonucleotide of claim 9, wherein the antisense oligonucleotide linked to the trans-cyclooctene has a structure of Formula I′a or Formula I′b: 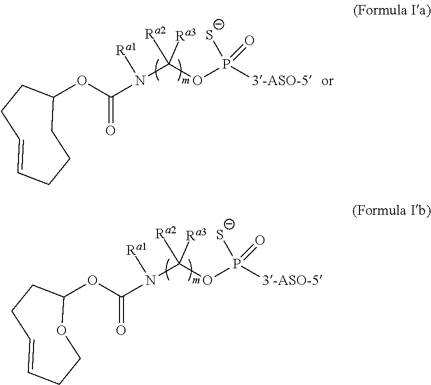
or a salt thereof.

17. The antisense oligonucleotide of any one of claims 8 to 16, wherein R^{a1} is H.

18. The antisense oligonucleotide of any one of claims 8 to 17, wherein R^{a2} is H.

19. The antisense oligonucleotide of any one of claims 8 to 17, wherein R^{a3} is H.

20. The antisense oligonucleotide of any one of claims 7 to 17, wherein R^{a2} and R^{a3} are H.

21. The antisense oligonucleotide of claim 8, wherein the antisense oligonucleotide linked to the trans-cyclooctene has a structure of Formula Ic or Formula Id: 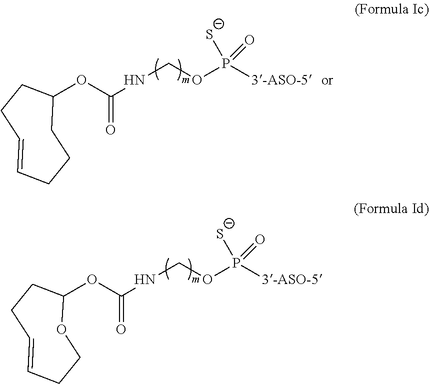
or a salt thereof.

22. The antisense oligonucleotide of claim 9, wherein the antisense oligonucleotide linked to the trans-cyclooctene has a structure of Formula I′c or Formula I′d: 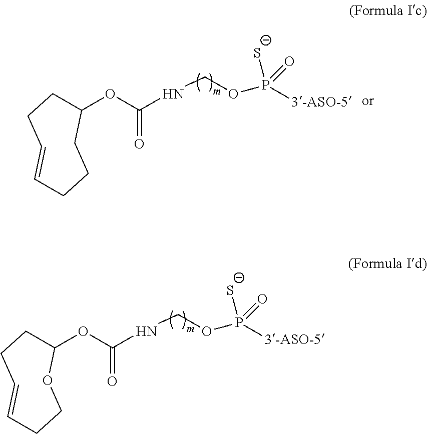
or a salt thereof.

23. The antisense oligonucleotide of any one of claims 8 to 22, wherein m is 1, 2, 3, 4, 5, or 6.

24. The antisense oligonucleotide of any one of claims 8 to 23, wherein m is 6.

25. The antisense oligonucleotide of claim 8, wherein the antisense oligonucleotide linked to the trans-cyclooctene has the following structure: 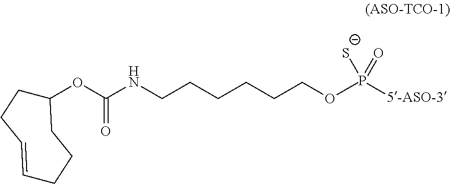
or a salt thereof.

26. The antisense oligonucleotide of claim 9, wherein the antisense oligonucleotide linked to the trans-cyclooctene has the following structure: 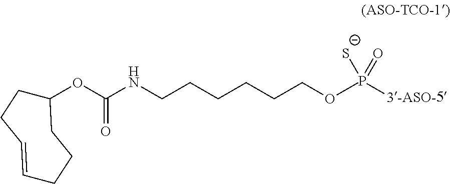
or a salt thereof.

27. A compound of Formula II: 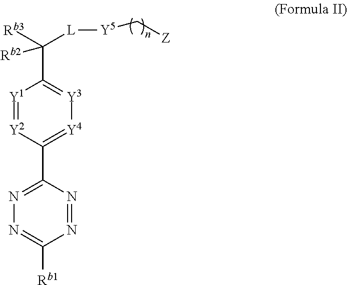
or a salt thereof, wherein
Z is ¹⁸F, ¹¹C-moiety, or chelated ⁶⁸Ga;
Y¹, Y², Y³, and Y⁴ are each independently CH or N;
L is C(O)NR^{b4} NR^{b4}C(O), O, OCH₂, NR^{b5}, or NR^{b5}CH₂;
Y⁵ is a bond, 5-6 membered heteroaryl or phenyl;
R^{b1} is H, C₁₋₆ alkyl, C₁₋₆ haloalkyl, pyridinyl, or pyrimidinyl;
R^{b2}, R^{b3}, R^{b4}, and R^{b5} are each independently H or C₁₋₆ alkyl; and
n is 0, 1, 2, 3, 4, 5, 6, 7, 8, 9 or 10.

28. The compound of claim 27, wherein Y⁵ is a bond.

29. The compound of claim 27, wherein Y⁵ is 5-6 membered heteroaryl.

30. The compound of claim 27, wherein Y⁵ is 5-membered heteroaryl.

31. The compound of claim 27, wherein Y⁵ is phenyl.

32. The compound of claim 27 having Formula IIa, Formula IIb, or Formula IIc: 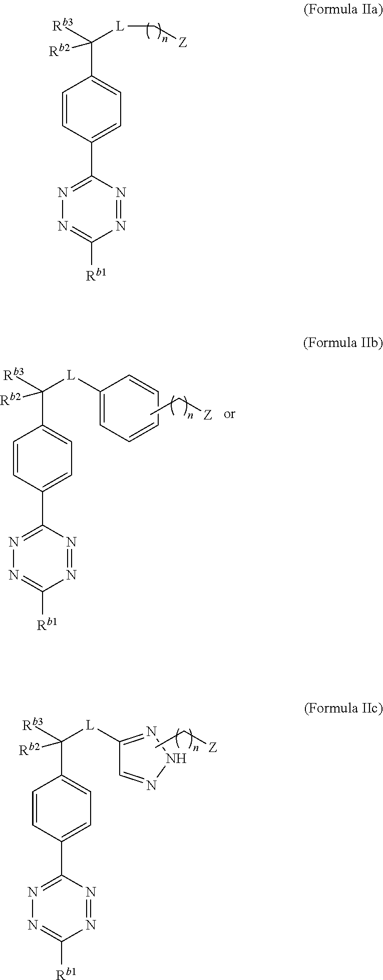
or a salt thereof.

33. The compound of any one of claims 27 to 32, wherein L is C(O)NR^{b4}.

34. The compound of any one of claims 27 to 32, wherein L is NR^{b4}C(O).

35. The compound of any one of claims 27 to 32, wherein L is O.

36. The compound of any one of claims 27 to 32, wherein L is OCH₂.

37. The compound of any one of claims 27 to 32, wherein L is NR^{b5}CH₂.

38. The compound of claim 27 having Formula IId, Formula IIe, Formula IIf, or Formula IIg: 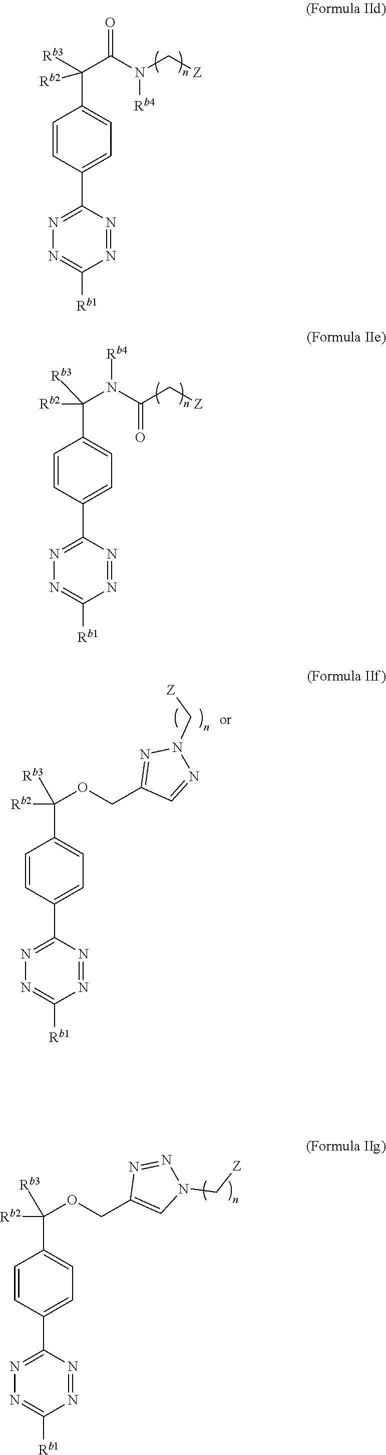
or a salt thereof.

39. The compound of any one of claims 27 to 38, wherein R^{b1} is H or C₁₋₆ alkyl.

40. The compound of any one of claims 27 to 38, wherein R^{b1} is H.

41. The compound of any one of claims 27 to 40, wherein R^{b2} is H.

42. The compound of any one of claims 27 to 40, wherein R^{b3} is H.

43. The compound of any one of claims 27 to 40, wherein R^{b2} and R^{b3} are H.

44. The compound of any one of claims 27 to 43, wherein R^{b4} is H.

45. The compound of any one of claims 27 to 44, wherein R^{b5} is H.

46. The compound of any one of claims 27 to 44, wherein R^{b5} is C₁₋₆ alkyl.

47. The compound of any one of claims 27 to 46, wherein n is 0, 1, 2, 3, or 4.

48. The compound of any one of claims 27 to 46, wherein n is 2.

49. The compound of any one of claims 27 to 46, wherein n is 0.

50. The compound of claim 27, wherein the compound is selected from:

| Compound no | Structure |
|---|---|
| 1 | 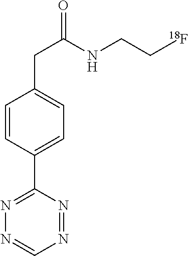 [SMILES: O=C(Cc1ccc(-c2nncnn2)cc1)NCC[18F]] |
| 2 | 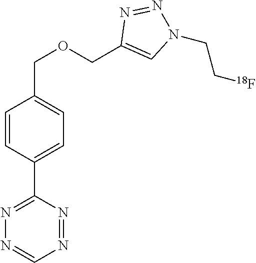 [SMILES: [18F]CCn1cc(COCc2ccc(-c3nncnn3)cc2)nn1] |
| 3 | 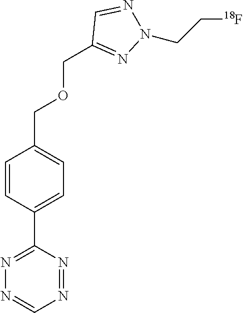 [SMILES: [18F]CCn1ncc(COCc2ccc(-c3nncnn3)cc2)n1] |
| 4 | 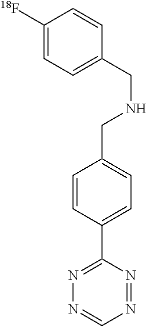 [SMILES: [18F]c1ccc(CNCc2ccc(-c3nncnn3)cc2)cc1] |
| 5 | 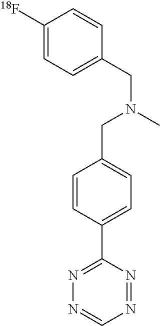 [SMILES: CN(Cc1ccc([18F])cc1)Cc1ccc(-c2nncnn2)cc1] |
| 6 | 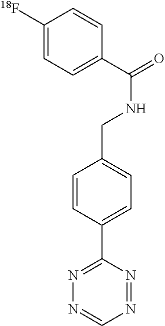 [SMILES: O=C(NCc1ccc(-c2nncnn2)cc1)c1ccc([18F])cc1] |
| 7 | 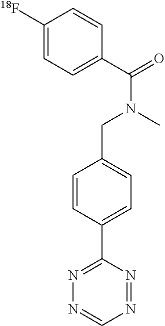 [SMILES: CN(Cc1ccc(-c2nncnn2)cc1)C(=O)c1ccc([18F])cc1] |
| | and |
| 8 | 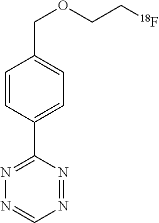 [SMILES: [18F]CCOCc1ccc(-c2nncnn2)cc1] |

or a salt thereof.

51. A pharmaceutical composition comprising the antisense oligonucleotide linked to a trans-cyclooctene of any one of claims 1 to 26 or the compound of any one of claims 27 to 50.

52. A process of preparing the Compound 1 of claim 50, wherein the process comprises: reacting 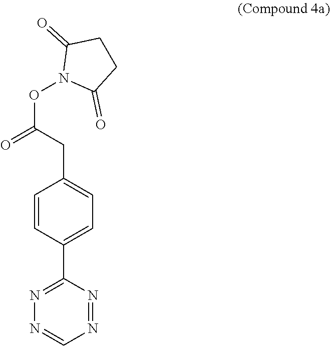 [SMILES: O=C(Cc1ccc(-c2nncnn2)cc1)ON1C(=O)CCC1=O]
with 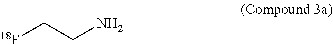
to provide Compound 1.

53. The process of claim 52, wherein Compound 3a is prepared by a process comprising reducing 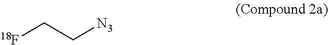
in the presence of a reducing agent to provide Compound 3a.

54. The process of claim 53, wherein Compound 2a is prepared by a process comprising converting 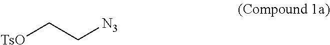 [SMILES: Cc1ccc(S(=O)(=O)OCCN=[N+]=[N-])cc1]
in the presence of ¹⁸F⁻ and K₂₂₂/K₂CO₃.

55. A method of determining the distribution of an antisense oligonucleotide in a subject, the method comprising in order:
(i) administering an antisense oligonucleotide linked to a trans-cyclooctene to the subject;
(ii) administering a radiolabeled tetrazine to the subject; and
(iii) imaging the distribution of the antisense oligonucleotide in the subject.

56. A method of determining the distribution of an antisense oligonucleotide in the brain and/or spinal cord of a subject, the method comprising in order:
(i) administering an antisense oligonucleotide linked to a trans-cyclooctene to the subject;
(ii) administering a central nervous system penetrant radiolabeled tetrazine to the subject; and
(iii) imaging the distribution of the antisense oligonucleotide in the brain and/or spinal cord of the subject.

57. A method of determining the concentration of an antisense oligonucleotide in the brain and/or spinal cord of a subject, the method comprising in order:
(i) administering an antisense oligonucleotide linked to a trans-cyclooctene to the subject;
(ii) administering a central nervous system penetrant radiolabeled tetrazine to the subject;
(iii) assessing the concentration of the antisense oligonucleotide in the brain and/or spinal cord of the subject.

58. The method of any one of claims 55 to 57, wherein the tetrazine is radiolabeled with a radiolabel selected from the group consisting of fluorine-18, carbon-11, and gallium-68.

59. The method of any one of claims 55 to 57, wherein the tetrazine is radiolabeled with fluorine-18.

60. The method of any one of claims 55 to 59, wherein the tetrazine is a compound of any one of claims 22 to 45.

61. The method of any one of claims 55 to 60, wherein the antisense oligonucleotide linked to trans-cyclooctene is the antisense oligonucleotide of any one of claims 2 to 21.

62. The method of any one of claims 55 to 61, wherein the antisense oligonucleotide linked to trans-cyclooctene is administered by intrathecal injection.

63. The method of any one of claims 55 to 62, wherein the radiolabeled tetrazine is administered by intravenous injection.

64. The method of any one of claims 55 to 63, wherein the radiolabeled tetrazine is administered about 24 hours after the administration of the antisense oligonucleotide linked to trans-cyclooctene.

65. The method of any one of claims 55 to 64, wherein the imaging is performed by PET/CT.

66. The method of any one of claims 55 to 64, wherein the imaging is performed by SPECT.

67. The method of any one of claims 55 to 66, wherein the subject is human.


SEQUENCE LISTING (2 sequences)

SEQ ID NO: 1 - DNA, 20 bp, Artificial Sequence
  source: /note=“Description of Artificial Sequence Synthetic oligonucleotide”
  modified_base (1)..(1): 2′-O-(2-methoxyethyl) modified nucleoside
  misc_feature (1)..(2): /note=“phosphorothioate internucleoside linkage”
  modified_base (2)..(2): 2′-O-(2-methoxyethyl) modified 5-methyl- cytosine
  misc_feature (2)..(5): /note=“phosphodiester internucleoside linkage”
  modified_base (3)..(3): 2′-O-(2-methoxyethyl) modified 5-methyl- cytosine
  modified_base (4)..(5): 2′-O-(2-methoxyethyl) modified nucleoside
  misc_feature (5)..(16): /note=“phosphorothioate internucleoside linkage”
  modified_base (7)..(7): 5-methyl-cytosine
  modified_base (16)..(16): 2′-O-(2-methoxyethyl) modified nucleoside
  misc_feature (16)..(18): /note=“phosphodiester internucleoside linkage”
  modified_base (17)..(17): 2′-O-(2-methoxyethyl) modified 5-methyl- cytosine
  modified_base (18)..(18): 2′-O-(2-methoxyethyl) modified 5-methyl-uracil
  misc_feature (18)..(20): /note=“phosphorothioate internucleoside linkage”
  modified_base (19)..(19): 2′-O-(2-methoxyethyl) modified 5-methyl- cytosine
  modified_base (20)..(20): 2′-O-(2-methoxyethyl) modified nucleoside
gccaggctggttatgacuca

SEQ ID NO: 2 - DNA, 20 bp, Artificial Sequence
  source: /note=“Description of Artificial Sequence Synthetic oligonucleotide”
  misc_feature (1)..(2): /note=“phosphorothioate internucleoside linkage”
  modified_base (1)..(5): 2′-O-(2-methoxyethyl) modified nucleoside
  misc_feature (2)..(5): /note=“phosphodiester internucleoside linkage”
  misc_feature (5)..(16): /note=“phosphorothioate internucleoside linkage”
  misc_feature (16)..(18): /note=“phosphodiester internucleoside linkage”
  modified_base (16)..(20): 2′-O-(2-methoxyethyl) modified nucleoside
  misc_feature (18)..(20): /note=“phosphorothioate internucleoside linkage”
gccaggctggttatgactca